(19) United States
(12) Patent Application Publication
(10) Pub. No.: US 2024/0168034 A1
(43) Pub. Date: May 23, 2024

(54) METHODS AND SYSTEMS FOR SINGLE CELL PROTEIN ANALYSIS

(71) Applicant: BOARD OF REGENTS, THE UNIVERSITY OF TEXAS SYSTEM, Austin, TX (US)

(72) Inventors: Eric V. ANSLYN, Austin, TX (US); Edward MARCOTTE, Austin, TX (US); Jagannath SWAMINATHAN, Austin, TX (US); Cecil J. HOWARD, II, Austin, TX (US); Angela BARDO, Austin, TX (US); Vy Que Thanh DANG, Austin, TX (US); Brendan FLOYD, Austin, TX (US); Wisath SAE-LEE, Austin, TX (US)

(21) Appl. No.: 18/518,877

(22) Filed: Nov. 24, 2023

Related U.S. Application Data
(63) Continuation of application No. PCT/US2022/031022, filed on May 26, 2022.
(60) Provisional application No. 63/193,328, filed on May 26, 2021.

(51) Int. Cl. G01N 33/68; G01N 33/543; G01N 33/58

(52) U.S. Cl. CPC G01N 33/6818; G01N 33/54313; G01N 33/54353; G01N 33/582

(57) ABSTRACT

Methods and compositions for partitioning and analyzing cells are provided herein. The partitioning methods include encapsulating single cells in droplets, enabling biomolecule analysis at the single cell level. Further to this concept, multiple biomarkers can be quantified from single red blood cells, including methods which may determine whether a subject has undergone autologous blood transfer.

### CROSS-REFERENCE

This application is a continuation of International Application No. PCT/US2022/031022, filed May 26, 2022, which claims the benefit of U.S. Provisional Application No. 63/193,328, filed May 26, 2021, each of which is incorporated herein by reference in their entirety.

### GOVERNMENT RIGHTS

This invention was made with government support under Grant No. R35 GM122480 awarded by the National Institutes of Health. The government has certain rights in the invention.

### BACKGROUND

Many diagnostic methods determine sample-level averages, and thus are blind to sub-population and/or cell-level variations within a sample. For example, mass spectrometric, enzyme-linked immunosorbent assay (ELISA), and immunohistological analyses often provide a single abundance value per target, regardless of the complexity or heterogeneity of the sample. Such measurements may be blind to a wide range of conditions, spanning from diseases such as cancer to doping in sports.

### SUMMARY

Various aspects of the present disclosure provide a method comprising: (a) contacting a cell and a support in a droplet, wherein the cell comprises at least one biomolecule; (b) labeling the at least one biomolecule; and (c) detecting a level of post-translational modification of the cell by detecting the at least one labeled biomolecule by sequencing by degradation. An aspect of the present disclosure provides a method comprising: (a) providing a droplet comprising a cell and a bead, wherein the cell comprises a polypeptide; (b) permeabilizing the cell within the droplet, thereby bringing the polypeptide in contact with the bead, wherein upon the polypeptide coming in contact with the bead, the polypeptide is coupled to the bead; (c) releasing the bead having the polypeptide coupled thereto from the droplet; and (d) identifying the polypeptide while still coupled to the bead. An aspect of the present disclosure provides a method comprising: (a) providing a droplet comprising a cell and a bead, wherein the cell comprises a polypeptide; (b) permeabilizing the cell within the droplet, thereby bringing the polypeptide in contact with the bead, wherein upon the polypeptide coming in contact with the bead, the polypeptide is coupled to the bead; (c) releasing the bead having the polypeptide coupled thereto from the droplet; and (d) identifying the polypeptide using sequencing by degradation

An aspect of the present disclosure provide a method comprising: (a) contacting a cell and a support in a droplet, wherein the cell comprises at least one biomolecule, wherein the at least one biomolecule comprises a first reactive moiety; wherein the support comprises a second reactive moiety; wherein the first reactive moiety and the second reactive moiety form a covalent bond to form a conjugate comprising the support and the at least one biomolecule; (b) collecting the conjugate into a container; (c) labeling the at least one biomolecule with at least one detectable moiety to produce at least one labeled biomolecule; and (d) detecting the at least one labeled biomolecule.

An aspect of the present disclosure provides a method comprising: (a) contacting a cell and a support in a droplet, wherein the cell comprises at least one polypeptide, wherein the contacting forms a conjugate comprising the support and the at least one polypeptide; (b) labeling the at least one polypeptide with at least one detectable moiety to produce at least one labeled polypeptide; and (c) detecting the at least one labeled polypeptide using sequencing by degradation.

In some embodiments, the droplet comprises one cell and one support. In some embodiments, the droplet is a water-in-oil droplet. In some embodiments, the droplet is a water-in-oil droplet. In some embodiments, the droplet has a mean volume of from about 0.5 pL to about 750 pL. In some embodiments, the droplet further comprises a cell lysis solution. In some embodiments, the droplet further comprises a buffer. In some embodiments, the at least one biomolecule is a polypeptide. In some embodiments, the at least one biomolecule is an intracellular peptide ore a secreted peptide. In some embodiments, the at least one biomolecule is a protein. In some embodiments, the at least one biomolecule is an intracellular protein or a secreted protein. In some embodiments, the at least one biomolecule is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), an oxidized protein, and a glycosylated protein.

In some embodiments, the cell is a red blood cell. In some embodiments, the support is a bead. In some embodiments, the bead comprises a barcode. In some embodiments, the barcode is an oligopeptide comprising from about 2 amino acids to about 30 amino acids. In some embodiments, the barcode comprises a non-natural amino acid. In some embodiments, the bead comprises a mean diameter of from about 15 nm to about 5 μm.

In some embodiments, the first reactive moiety comprises a sulfhydryl group. In some embodiments, the first reactive moiety comprises a cysteine side chain. In some embodiments, the first reactive moiety comprises a histidyl side chain. In some embodiments, the second reactive moiety comprises a pyridine carboxyaldehyde. In some embodiments, the second reactive moiety comprises a maleimide group. In some embodiments, the second reactive moiety comprises a haloacetyl group. In some embodiments, the second reactive moiety comprises a pyridyl disulfide. In some embodiments, the support comprises a plurality of second reactive moieties. In some embodiments, the covalent bond is a disulfide bond. In some embodiments, the covalent bond is a thioether bond.

In some embodiments, the collecting comprises adding a reagent, wherein the reagent releases the conjugate from the droplet. In some embodiments, the labeling comprises immunostaining. In some embodiments, the detectable moiety comprises a fluorescent moiety. In some embodiments, the fluorescent moiety comprises an antibody. In some embodiments, the fluorescent moiety comprises a fluorescent dye. In some embodiments, the labeling produces a plurality of labeled biomolecules. In some embodiments, the detecting comprises sequencing by degradation.

Another aspect of the present disclosure provides a non-transitory computer readable medium comprising machine executable code that, upon execution by one or more computer processors, implements an of the methods above or elsewhere herein.

Another aspect of the present disclosure provides a system comprising one or more computer processors and computer memory coupled thereto. The computer memory comprises machine executable code that, upon execution by one or more computer processors, implements any of the methods above or elsewhere herein.

Additional cases and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 provides a microscope image of a microfluidic junction in which water-in-oil droplets form around an average of one cell and one bead.

FIG. 2 provides a microscope image of a red blood cell 201 and a bead 202 suitable for methods of the present disclosure. 203 illustrates maleimide functionalization of streptavidin beads.

FIG. 3 illustrates a scheme for an assay to identify biomolecule levels from single cells.

FIG. 4 illustrates a reactive site and capture moiety 402 functionalized bead, along with fluorescence microscope images depicting green fluorescent protein (GFP) capture on non-capture moiety functionalized beads 404 and capture moiety functionalized beads 405.

FIGS. 5A-5D illustrates a design for a microfluidic chip used in methods of the disclosure. FIG. 5A shows a fabricated microfluidic chip, held above a normal sized computer keyboard for scale; FIG. 5B provides an image of water-in-oil droplets forming at a 4-way junction in the microfluidic chip; FIG. 5C provides a further image of the 4-way junction from FIG. 5B, in which a bead 506 may be discerned in a droplet 505; FIG. 5D provides a double 4-way-junction schematic for forming droplets comprising an average of one cell and one bead.

FIG. 6 outlines the contents of multiple HbA1c bead calibrant sets.

FIGS. 7A-7C depict two separate human populations (top) with different red blood cell HbA1c:HbA ratios (bottom graphs). FIG. 7C shows HbA1c measurements on a mixture of blood with low HbA1c levels from the human population of FIG. 7A and blood with high HbA1c levels from the human population of FIG. 7B.

FIG. 8 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and/or substitutions may occur to those skilled in the art without departing from the invention. It should be understood that various alternatives to the embodiments of the invention described herein may be employed.

While many conditions manifest in sub-population level aberrations, for example affecting no more than 10%, 1%, or 0.1% of a particular type of cell in a subject, monitoring changes at the single cell level exceeds the current limits of many diagnostic methods. Thus, novel strategies for isolating and/or measuring the contents of single cells are needed to address the growing demands of personalized diagnostic monitoring. Disclosed herein are methods and/or systems directed to measure a quantity or expression level of an analyte (e.g., a biomolecule) in a sub-population of cells obtained from a sample instead of measuring a global quantity or average expression level of the analyte in the sample. The methods and/or systems disclosed herein can also measure a ratio of quantities or expression levels of multiple analytes (e.g., biomolecules) in a sub-population of cells obtained from a sample.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “biomolecule” as used herein generally refers to any biomolecule associated with a cell. In some examples, a biomolecule is an intracellular molecule of a cell, a secreted biomolecule of a cell, or a metabolite of a cell. In some examples, a biomolecule is a peptide. In some examples, a biomolecule is a protein. In some examples, a biomolecule is an intracellular protein of a cell, a secreted protein of a cell, or a protein metabolite of a cell. In some examples, a biomolecule is an intracellular peptide of a cell, a secreted peptide of a cell, or a peptide metabolite of a cell.

The terms “polypeptide” and “peptide,” as used interchangeably herein, generally to refer to a polymer comprising amino acids in which an amino acid may be linked to another amino acid by a peptide bond. In some examples, a polypeptide is a protein. The amino acid may be a naturally occurring amino acid or a non-naturally occurring amino acid (e.g., an amino acid analogue). The polypeptide may be linear or branched. The polypeptide may include modified amino acids. The polypeptide may be interrupted by non-amino acids. A polypeptide may occur as a single chain or an associated chain. The polypeptide may include a plurality of amino acids. The polypeptide may have a secondary and tertiary structure (e.g., the polypeptide may be a protein). In some examples, the polypeptide comprises at least 2, 3, 4, 5, 6, 7, 8, 9, 10, 20, 30, 40, 50, 100, 1000, 10,000, or more amino acids. The polypeptide may be a fragment of a larger polymer. In some examples, the polypeptide is a fragment of a larger polypeptide, such as a fragment of a protein.

The term “amino acid,” as used herein, generally refers to a naturally occurring or non-naturally occurring amino acid (e.g., an amino acid analogue). The non-naturally occurring amino acid may be an engineered or synthesized amino acid. An amino acid may contain a “side chain”, which may differentiate amino acid types from one another.

The terms “amino acid sequence,” “peptide sequence,” and “polypeptide sequence,” as used herein, generally refer to a sequence of at least two amino acids or amino acid analogs that are covalently linked (e.g., by a peptide (amide) bond or an analog of a peptide bond). A peptide sequence may refer to a complete sequence or a portion of a sequence. For example, a peptide sequence may contain gaps, positions with unknown identities, or positions that may accommodate distinct species.

As used herein, the term “side chain” generally refers to a structure attached to an alpha carbon (attaching an amine and/or a carboxylic acid group of an amino acid) that may be unique to each type of amino acid. A side chain may have a certain shape, size, charge, reactivity, or a combination thereof. A side chain may contain a basic moiety (e.g., the guanidino group in arginine), an acidic moiety (e.g., the carboxylic acid in aspartic acid), a polar moiety (e.g., the hydroxyl groups in serine, threonine, and tyrosine), a hydrophobic moiety (e.g., the alkyl groups in leucine, isoleucine, alanine, and valine), or any combination thereof. In some cases, an amino acid contains more than one side chain. The side chain may be or include hydrogen, an alkyl group, a hydroxyl group, an aryl group, a heteroaryl group, a carboxylic acid, an amide, an amine, a guanidine, a thiol, a thioether, a selenol, or any combination thereof. In some instances, the side chain is a hydrogen (an amino acid with a hydrogen side chain may be, e.g., glycine).

As used herein, the term “Edman degradation” generally refers to a method of removing an amino acid from the N-terminal end of a polypeptide using an isothiocyanate (e.g., phenyl isothiocyanate). Edman degradation may be coupled with various polypeptide sequencing and/or analysis methods. Edman degradation may be performed sequentially.

As used herein, the term “array” generally refers to a population of sites. Such populations of sites may be differentiated from one another according to relative location. Different molecules that are at different sites of an array may be differentiated from each other according to the locations of the sites in the array. An individual site of an array may include one or more molecules of a particular type. For example, a site may include a single polypeptide having a particular sequence or a site may include several polypeptides having the same sequence. The sites of an array may be different features located on the same substrate. Such features may include, without limitation, wells in a substrate, beads (or other particles) in or on a substrate, projections from a substrate, ridges on a substrate or channels in a substrate. The sites of an array may be separate substrates each bearing at least one molecule. Different molecules attached to separate substrates may be identified according to the locations of the substrates on a surface to which the substrates are associated or according to the locations of the substrates in a liquid or gel. Such different molecules may have the same or different sequences. An array may include one or more wells, and a well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that may couple to a reactive group. The label may comprise at least one reactive group (e.g., a first reactive group and a second reactive group). The at least one reactive group may be configured to couple to a polypeptide. The at least one reactive group may be configured to couple to a support. The at least one reactive group may be configured to couple to a reporter moiety. A label may provide a measurable signal.

As used herein, the term “polymer matrix” generally refers to a (e.g., continuous) phase material that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and/or crosslinked polymer units. A polymer matrix may also contain non-polymeric species intercalated within its interstitial spaces not occupied by polymer chains. The intercalated species may be solid, liquid, or gaseous species. For example, the term ‘polymer matrix’ may encompass desiccated hydrogels, hydrated hydrogels, and/or hydrogels containing glass fibers.

The term “reporter moiety,” as used herein, generally refers to an agent that generates a measurable signal. Such a signal may include, but is not limited to, fluorescence (e.g., a dye), visible light, motion (e.g., a mass tag), radiation, or a nucleic acid sequence (e.g., a barcode). Such a signal may include, but is not limited to, fluorescence, phosphorescence, or, radiation. Such signal may be light (or electromagnetic radiation). The light may include a frequency or frequency distribution in the visible portion of the electromagnetic spectrum. For example, the light may be infrared or ultraviolet light. The signal may be an electrostatic, a conductive, or an impedance signal. The signal may be a charge. A “reporter” may comprise a “reporter moiety”. The reporter may comprise a reactive group. The reactive group may be configured to couple to a label.

The term “sequencing by degradation”, as used herein, refers to a method for analyzing a biomolecule comprising: (a) providing a polypeptide, wherein the polypeptide comprises at least one labeled internal amino acid; (b) detecting at least one signal or signal change from the polypeptide to identify at least a portion of a sequence of the polypeptide; and (c) subjecting the polypeptide to conditions sufficient to remove at least one amino acid from the polypeptide. In some embodiments, the polypeptide is immobilized to a support. In some embodiments, at least one amino acid is removed from an N-terminus of the polypeptide. In some embodiments, subsequent to (c), the at least one labeled internal amino acid becomes a labeled terminal amino acid. In some embodiments, the at least one labeled internal amino acid is from a plurality of labeled amino acids, wherein at least one signal or signal change comprises a collective signal from the plurality of labeled amino acids. In some embodiments, the plurality of labeled amino acids comprise amino acids with different labels. In some embodiments, the different labels generate signals with different signal patterns. In some embodiments, the at least one labeled internal amino acid comprises one or more members selected from the group consisting of lysine, glutamate, and aspartate. In some embodiments, the at least one labeled internal amino acid comprises an amino acid having a label covalently attached thereto, which label generates the at least one signal or signal change. In some embodiments, the at least one labeled internal amino acid comprises an amino acid having a dye coupled thereto, which dye generates the at least one signal or signal change. In some embodiments, the at least one signal or signal change is an optical signal.

In some embodiments, the at least one signal or signal change comprises a plurality of signals of different intensities. In some embodiments, the at least one signal or signal change comprises a plurality of signals of different frequencies or frequency ranges. In some embodiments, the at least one amino acid is removed from the polypeptide by a degradation reaction. In some embodiments, the degradation reaction is Edman degradation. In some embodiments, the at least one signal or signal change is detected with an optical detector having single-molecule sensitivity. In some embodiments, In some embodiments, the method further comprises processing at least a portion of the sequence against a reference sequence to identify the polypeptide or a protein from which the polypeptide is derived. In some embodiments, the method further comprises, subsequent to (c), (i) identifying the at least the portion of the sequence of the polypeptide to identify the polypeptide, and (ii) using the polypeptide identified in (i) to quantify the polypeptide or a protein from which the polypeptide was derived. In some embodiments, in (a), less than all amino acids of the polypeptide are labeled. In some embodiments, the method further comprises (i) repeating (b) and (c) to detect at least one additional signal or signal change from the polypeptide and (ii) using the at least one signal or signal change and the at least one additional signal or signal change to identify the at least the portion of the sequence.

As used herein, the term “fluorescence” refers to the emission of visible light by a substance that has absorbed light of a different wavelength. In some embodiments, fluorescence provides a non-destructive method of tracking and/or analyzing biological molecules based on the fluorescent emission at a specific wavelength. Proteins (including antibodies), peptides, nucleic acid, oligonucleotides (including single stranded and double stranded primers) may be “labeled” with a variety of extrinsic fluorescent molecules referred to as fluorophores. Isothiocyanate derivatives of fluorescein, such as carboxyfluorescein, are an example of fluorophores that may be conjugated to proteins (such as antibodies for immunohistochemistry) or nucleic acids. In some embodiments, fluorescein may be conjugated to nucleoside triphosphates and incorporated into nucleic acid probes (such as “fluorescent-conjugated primers”) for in situ hybridization. In some embodiments, a molecule that is conjugated to carboxyfluorescein is referred to as “FAM-labeled”.

As used herein, sequencing of peptides “at the single molecule level” refers to amino acid sequence information obtained from individual (i.e. single) peptide molecules in a mixture of diverse peptide molecules. It is not necessary that the present disclosure be limited to methods where the amino acid sequence information obtained from an individual peptide molecule is the complete or contiguous amino acid sequence of an individual peptide molecule. In some embodiment, it is sufficient that only partial amino acid sequence information is obtained, allowing for identification of the peptide or protein. Partial amino acid sequence information, including for example the pattern of a specific amino acid residue (i.e. Lysine) within individual peptide molecules, may be sufficient to uniquely identify an individual peptide molecule. For example, a pattern of amino acids such as X-X-X-Lys-X-X-X-X-Lys-X-Lys, which indicates the distribution of Lysine molecules within an individual peptide molecule, may be searched against a known proteome of a given organism to identify the individual peptide molecule. It is not intended that sequencing of peptides at the single molecule level be limited to identifying the pattern of Lysine residues in an individual peptide molecule; sequence information for any amino acid residue (including multiple amino acid residues) may be used to identify individual peptide molecules in a mixture of diverse peptide molecules.

As used herein, “single molecule resolution” refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). In one embodiment, this may include the ability to simultaneously record the fluorescent intensity of multiple individual (i.e. single) peptide molecules distributed across the glass surface. Optical devices are commercially available that can be applied in this manner. For example, a microscope equipped with total internal reflection illumination and an intensified charge-couple device (CCD) detector is available. Imaging with a high sensitivity CCD camera allows the instrument to simultaneously record the fluorescent intensity of multiple individual (i.e. single) peptide molecules distributed across a surface. In one embodiment, image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

As used herein, the term “collective signal” refers to the combined signal that results from the first and second labels attached to an individual peptide molecule. As used herein, the term “experimental cycle” refers to one round of single molecule sequencing, comprised of the Edman degradation of a single amino acid residue followed by TIRF measurement of fluorescence intensities.

As used herein, the term “Hertz” or “Hz” refers to the rate at which partitioning is formed. In some embodiments, the partitioning is forming a water-in-oil droplets. In some embodiments, the partitioning is forming an oil-in-water droplets. In some embodiments, partitions in the disclosure can be formed in units of Hz, or partitions/second. In some embodiments, droplets in the disclosure can be formed in units of Hz, or partitions/second.

### Methods and Systems for Isolating Cells

Disclosed herein is a method comprising: (a) contacting a cell and a support in a droplet, wherein said cell comprises at least one biomolecule, wherein said contacting forms a conjugate comprising said support and said at least one biomolecule; (b) collecting said conjugate into a container; (c) labeling said at least one biomolecule of said conjugate in said container with at least one detectable moiety to produce at least one labeled biomolecule; and (d) detecting said at least one labeled biomolecule. Disclosed herein is a method comprising: (a) contacting a cell and a support in a droplet, wherein said cell comprises at least one biomolecule, wherein said at least one biomolecule comprises a first reactive moiety; wherein said support comprises a second reactive moiety; wherein said first reactive moiety and said second reactive moiety form a covalent bond to form a conjugate comprising said support and said at least one biomolecule; (b) collecting said conjugate into a container; (c) labeling said at least one biomolecule with at least one detectable moiety to produce at least one labeled biomolecule; and (d) detecting said at least one labeled biomolecule.

Disclosed herein is a method comprising: (a) contacting a cell and a support in a droplet, wherein said cell comprises at least one polypeptide, wherein said contacting forms a conjugate comprising said support and said at least one biomolecule; (b) labeling said at least one biomolecule with at least one detectable moiety to produce at least one labeled biomolecule; and (c) detecting said at least one labeled biomolecule using sequencing by degradation. Disclosed herein is a method comprising: (a) sorting a biological sample to extract a subset of cells; (b) contacting at least one biomolecule from said subset of cells and a support in a droplet, wherein said cell comprises said at least one biomolecule, wherein said contacting forms a conjugate comprising said support and said at least one biomolecule; (c) after said contacting, collecting said conjugate into a container; (d) after said collecting, labeling said at least one biomolecule in said container with at least one detectable moiety to produce at least one labeled biomolecule; and (e) detecting said at least one detectable moiety. Disclosed herein is a composition comprising: (a) at least one polypeptide; and (b) a singular support, wherein said at least one biomolecule and said singular support are coupled together by a covalent bond, and wherein said at least one biomolecule and said singular support are enclosed in a droplet.

Disclosed herein is a method comprising: (a) providing a droplet comprising a cell and a bead, wherein said cell comprises a polypeptide; (b) permeabilizing said cell within said droplet, thereby bringing said polypeptide in contact with said bead, wherein upon said polypeptide coming in contact with said bead, said polypeptide is coupled to said bead; (c) releasing said bead having said polypeptide coupled thereto from said droplet; and (d) identifying said polypeptide while still coupled to the bead. Disclosed herein is a method comprising: (a) providing a droplet comprising a cell and a bead, wherein said cell comprises a polypeptide; (b) permeabilizing said cell within said droplet, thereby bringing said polypeptide in contact with said bead, wherein upon said polypeptide coming in contact with said bead, said polypeptide is coupled to said bead; (c) releasing said bead having said polypeptide coupled thereto from said droplet; and (d) identifying said polypeptide using sequencing by degradation. Disclosed herein is a method comprising: (a) contacting a cell and a support in a droplet, wherein said cell comprises at least one biomolecule; (b) labeling said at least one biomolecule; and (c) detecting a level of post-translational modification of said cell by detecting said at least one labeled biomolecule by sequencing by degradation.

Various embodiments of the present disclosure provide means for separating or isolating a species or a portion of a sample. In many cases, such separating or isolating comprises collecting the species or the portion of a sample within a partition. In some embodiments, a species or a portion of a sample can be separated into a droplet. In some cases, a species or a portion of a sample can be separated into a well, a microwell, a continuous phase within an emulsion, a test tube, a spot, a compartment, or a capsule.

In some cases, a partition may isolate a species from its surroundings. In some cases, a partition may isolate a portion of a sample from its surroundings. For example, a partition may comprise a solid barrier (e.g., a test tube wall) that prevents influx or efflux of species from the partition. A partition may also partially isolate a species or a portion of a sample from its surroundings. In some embodiments, a partition can be a water-in-oil droplet, wherein the outer layer of the oil droplet is impermeable to proteins, but through which solvent molecules and/or small analytes may diffuse. As a further example, a water-in-oil droplet may prevent diffusion of large species (e.g., exosomes and/or proteins) and/or of charged species, while allowing diffusion of small species, such as steroids and/or polyketides.

In various cases, the methods of the present disclosure comprise partitioning, isolating, or separating a cell or biological species of interest. The cell or the biological species of interest may be derived from a sample comprising a plurality of cells or biological species, and the partitioning, isolating, or separating may separate or isolate the cell or the biological species from the sample. In some cases, a partition may comprise a cell. In some cases, a partition may comprise a biological species of interest. In some cases, a partition may comprise a capture reagent, such as a capture moiety functionalized bead. In some cases, a partition may comprise multiple capture reagents, such as multiple capture moiety functionalized beads. A partition may comprise a cell or a biological species of interest and/or a capture reagent (e.g., a bead). In some cases, the cell or the biological species of interest is partitioned with a reagent (e.g., a reagent for cellular or nuclear lysis; or a reagent for biomolecule detection). In some cases, the cell or the biological species of interest is partitioned without a reagent.

In some embodiments, a partition or droplet may be contained within a liquid medium. In some embodiments, the droplet can be a water-in-oil droplet suspended in an aqueous solution. In some embodiments, a water-in-oil droplet can be suspended in an aqueous solution to form an emulsion.

Methods for droplet generation consistent with the present disclosure include flow focusing (e.g., microfluidic flow focusing or hydrodynamic flow focusing), co-flowing droplet formation, cross-flowing droplet formation, droplet fusion, and/or droplet splitting. For example, a water-in-oil droplet may be generated by oil injection (e.g., through a nozzle) into water. The droplet may be generated in using a microfluidic or nanofluidic flow device. In such cases, a droplet may be generated at a confluence between two or more channels (e.g., a junction), for example by injection or flow of a first liquid into a second liquid. In some cases, a reagent flows into a junction through a first channel and a cell flows through a junction through a second channel. In some cases, a reagent flows into a junction through a first channel, a cell flows through a junction through a second channel, and/or the droplet medium (e.g., an oil) flows into the junction through a third channel. In some cases, a junction comprises an outflow channel through which a mature droplet may flow. In some embodiments, the size of a droplet may be controlled by fluid velocities and/or channel sizes. In some cases, a droplet containing a cell is merged with a droplet containing a bead to form a droplet containing a cell and a bead.

In some cases, a fluidic channel or plurality of fluidic channels may be used to form a droplet. In some cases, the droplet forms at the confluence point of a plurality of channels. In some cases, the diameter of the fluidic channel may affect the size of a droplet. In some cases, the fluidic channel comprises a diameter of from about 10 μm to about 100 μm, from about 100 m to about 150 μm, from about 150 μm to about 200 μm, from about 200 μm to about 250 am, from about 250 μm to about 300 am, from about 300 μm to about 350 μm, from about 350 μm to about 400 μm, from about 400 μm to about 450 μm, from about 450 μm to about 500 am, from about 500 μm to about 600 μm, from about 600 μm to about 700 am, from about 700 μm to about 800 μm, from about 800 μm to about 900 μm, or from about 900 μm to about 1000 am. In some cases, the fluidic channel comprises a diameter of at least about 1000 am, at least about 750 μm, at least about 500 μm, at least about 400 μm, at least about 300 am, at least about 200 m, at least about 100 μm, at least about 50 μm, at least about 25 μm, or at least about 10 μm. In some cases, the fluidic channel comprises a diameter of about 1000 μm, about 750 μm, about 500 μm, about 400 μm, about 300 μm, about 200 μm, about 100 μm, about 50 μm, about 25 μm, or about 10 μm. In some cases, the fluidic channel comprises a diameter of at most about 1000 m, at most about 750 μm, at most about 500 μm, at most about 400 μm, at most about 300 μm, at most about 200 μm, at most about 100 μm, at most about 50 μm, at most about 25 μm, or at most about 10 μm.

The partitioning may be performed at a variety of rates. In some embodiments, the partitioning is performed at a rate of at from about 5 Hz to about 1,000 Hz. In some embodiments, the partitioning is performed at a rate of at from about 5 Hz to about 50 Hz, from about 50 Hz to about 100 Hz, from about 100 Hz to about 150 Hz, from about 150 Hz to about 200 Hz, from about 200 Hz to about 250 Hz, from about 250 Hz to about 300 Hz, from about 300 Hz to about 350 Hz, from about 350 Hz to about 400 Hz, from about 400 Hz to about 450 Hz, from about 450 Hz to about 500 Hz, from about 500 Hz to about 600 Hz, from about 600 Hz to about 700 Hz, from about 700 Hz to about 800 Hz, from about 800 Hz to about 900 Hz, or from about 900 Hz to about 1,000 Hz. In some embodiments, the partitioning is performed at a rate of at least about 5 Hz, at least about 50 Hz, at least about 100 Hz, at least about 150 Hz, at least about 200 Hz, at least about 250 Hz, at least about 300 Hz, at least about 350 Hz, at least about 400 Hz, at least about 450 Hz, at least about 500 Hz, at least about 550 Hz, at least about 600 Hz, at least about 650 Hz, at least about 700 Hz, at least about 750 Hz, at least about 800 Hz, at least about 850 Hz, at least about 900 Hz, at least about 950 Hz, or at least about 1,000 Hz. In some embodiments, the partitioning is performed at a rate of about 5 Hz, about 50 Hz, about 100 Hz, about 150 Hz, about 200 Hz, about 250 Hz, about 300 Hz, about 350 Hz, about 400 Hz, about 450 Hz, about 500 Hz, about 550 Hz, about 600 Hz, about 650 Hz, about 700 Hz, about 750 Hz, about 800 Hz, about 850 Hz, about 900 Hz, about 950 Hz, or about 1,000 Hz. In some embodiments, the partitioning is performed at a rate of at most about 5 Hz, at most about 50 Hz, at most about 100 Hz, at most about 150 Hz, at most about 200 Hz, at most about 250 Hz, at most about 300 Hz, at most about 350 Hz, at most about 400 Hz, at most about 450 Hz, at most about 500 Hz, at most about 550 Hz, at most about 600 Hz, at most about 650 Hz, at most about 700 Hz, at most about 750 Hz, at most about 800 Hz, at most about 850 Hz, at most about 900 Hz, at most about 950 Hz, or at most about 1,000 Hz.

The partitioning may be performed at a variety of rates. In some embodiments, the partitioning is performed at a rate of at from about 1,000 Hz to about 100,000 Hz. In some embodiments, the partitioning is performed at a rate of at from about 1,000 Hz to about 5,000 Hz; from about 5,000 Hz to about 10,000 Hz; from about 10,000 Hz to about 25,000 Hz; from about 25,000 Hz to about 50,000 Hz; from about 50,000 Hz to about 75,000 Hz; or from about 75,000 Hz to about 100,000 Hz. In some embodiments, the partitioning is performed at a rate of at from about 1,000 Hz to about 5,000 Hz. In some embodiments, the partitioning is performed at a rate of at from about 25,000 Hz to about 50,000 Hz. In some embodiments, the partitioning is performed at a rate of at from about 75,000 Hz to about 100,000 Hz.

In some embodiments, the partitioning is performed at a rate of at least about 1,000 Hz; at least about 5,000 Hz; at least about 10,000 Hz; at least about 25,000 Hz; at least about 50,000 Hz; at least about 75,000 Hz; or at least about 100,000 Hz. In some embodiments, the partitioning is performed at a rate of at least about 5,000 Hz. In some embodiments, the partitioning is performed at a rate of at least about 20,000 Hz. In some embodiments, the partitioning is performed at a rate of at least about 40,000 Hz. In some embodiments, the partitioning is performed at a rate of at least about 60,000 Hz. In some embodiments, the partitioning is performed at a rate of at least about 80,000 Hz. In some embodiments, the partitioning is performed at a rate of at most about 1,000 Hz; at most about 5,000 Hz; at most about 10,000 Hz; at most about 25,000 Hz; at most about 50,000 Hz; at most about 75,000 Hz; or at most about 100,000 Hz. In some embodiments, the partitioning is performed at a rate of at most about 5,000 Hz. In some embodiments, the partitioning is performed at a rate of at most about 20,000 Hz. In some embodiments, the partitioning is performed at a rate of at most about 40,000 Hz. In some embodiments, the partitioning is performed at a rate of at most about 60,000 Hz. In some embodiments, the partitioning is performed at a rate of at most about 80,000 Hz. In some embodiments, the partitioning is performed at a rate of about 1,000 Hz; about 5,000 Hz; about 10,000 Hz; about 25,000 Hz; about 50,000 Hz; about 75,000 Hz; or about 100,000 Hz. In some embodiments, the partitioning is performed at a rate of about 5,000 Hz. In some embodiments, the partitioning is performed at a rate of about 20,000 Hz. In some embodiments, the partitioning is performed at a rate of about 40,000 Hz. In some embodiments, the partitioning is performed at a rate of about 60,000 Hz. In some embodiments, the partitioning is performed at a rate of about 80,000 Hz.

A biological species (e.g. a cell) or a capture reagent may be added to a droplet, for example by contacting the droplet with a fluid comprising the cell and/or capture reagent. In some embodiments, the capture reagent is a bead. The partitioning may be performed at multiple locations (e.g., multiple droplet generating junctions). The partitioning may result in a range of partition occupancy levels. A partitioning method may yield a plurality of partitions, of which a subset may comprise exactly one cell and exactly one bead. The addition of cells or capture reagents to droplets may be controlled such that the droplets comprise a predetermined number of cells and/or capture reagents. In some cases, the addition of a cell and a capture reagent to a droplet results in from about 30% to about 40%, from about 40% to about 50%, from about 50% to about 60%, from about 60% to about 70%, from about 70% to about 80%, from about 80% to about 85%, from about 85% to about 90%, from about 90% to about 95%, or from about 95% to about 99.5% of droplets containing at least one cell and one capture reagent comprising exactly one cell and exactly one capture reagent. In some cases, the addition of a cell and a capture reagent to a droplet results in at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 99.5% of droplets containing at least one cell and one capture reagent comprising exactly one cell and exactly one capture reagent. In some cases, the addition of a cell and a capture reagent to a droplet results in about 30%, about 35% about 40%, about 45%, about 50%, about 55%, about 60%, about 65%, about 70%, about 75%, about 80%, about 85%, about 90%, about 95%, about 98%, about 99%, or about 99.5% of droplets containing at least one cell and one capture reagent comprising exactly one cell and exactly one capture reagent. In some cases, the addition of a cell and a capture reagent to a droplet results in at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, at most about 98%, at most about 99%, or at most about 99.5% of droplets containing at least one cell and one capture reagent comprising exactly one cell and exactly one capture reagent.

In some cases, the addition of a cell and a capture reagent to a droplet results in at most about 10%, at most about 5%, at most about 1%, at most about 0.1%, at most about 0.01%, or fewer than 0.001% of the droplets containing more than two cells or more than two capture reagents.

In some cases, a process for forming droplets may result in more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than about 95%, more than about 98%, more than about 99%, more than about 99.5%, more than about 99.75%, or more than about 99.99% of droplets which contain at least one cell and at least one bead. In some cases, a process for forming droplets may result in more than about 70%, more than about 75%, more than about 80%, more than about 85%, more than about 90%, more than about 95%, more than about 98%, more than about 99%, more than about 99.5%, more than about 99.75%, or more than about 99.99% of droplets which contain exactly one cell and exactly one bead. In some cases, a process for forming droplets may result in more than about 80% of droplets which contain at least one cell and at least one bead. In some cases, a process for forming droplets may result in more than about 90% of droplets which contain at least one cell and at least one bead. In some cases, a process for forming droplets may result in more than about 80% of droplets which contain exactly one cell and exactly one bead. In some cases, a process for forming droplets may result in more than about 90% of droplets which contain exactly one cell and exactly one bead.

In some cases, a process for forming droplets may result in about 10%, about 5%, about 1%, about 0.1%, about 0.01%, or about 0.001% of the droplets containing more than two cells and/or more than two beads. In some cases, a process for forming droplets may result in at most about 5% of the droplets containing more than two cells and/or more than two beads. In some cases, a process for forming droplets may result in at most about 10%, at most about 5%, at most about 1%, at most about 0.1%, at most about 0.01%, or at most about 0.001% of the droplets containing more than two cells and/or more than two beads. In some cases, a process for forming droplets may result in at most about 5% of the droplets containing more than two cells and/or more than two beads. In some cases, a process for forming droplets may result in at most about 1% of the droplets containing more than two cells and/or more than two beads. In some cases, only a subset of the formed droplets will contain at least one cell and at least one bead.

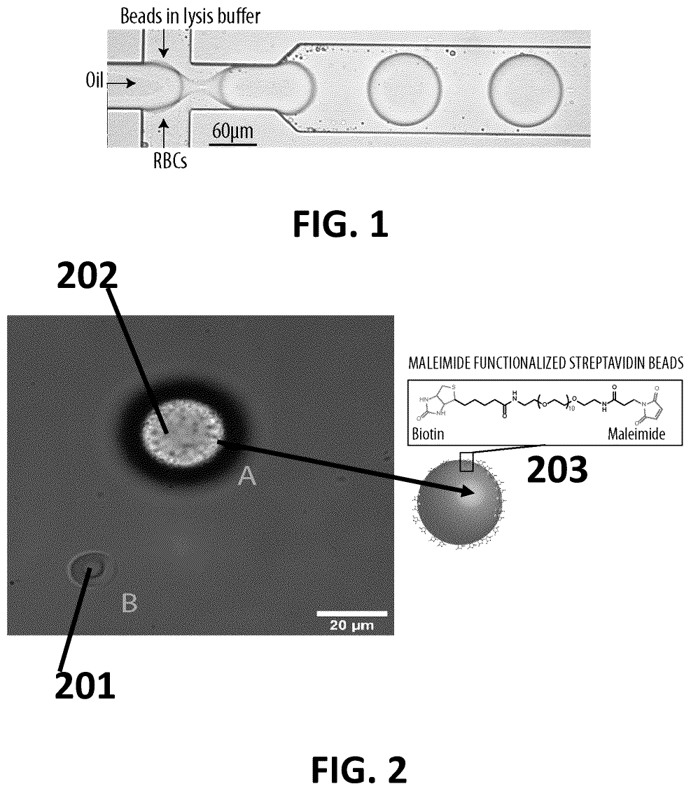

FIG. 1 provides an image of a water-in-oil droplet forming at a flow-focusing junction. Beads are introduced to an oil stream from the top flow stream, and red blood cells (RBCs) are introduced to the oil stream from the bottom junction. Water-in-oil droplets are formed at the junction and comprise a red blood cell and a bead. FIG. 2 shows a 100× DIC (Differential Interference Contrast) microscopic image of a red blood cell 201 and a streptavidin functionalized bead 202, which is coupled to a maleimide capture reagent by a biotin linker 203 on the surface of the streptavidin-functionalized bead.

In some cases, a partition may be a droplet. In some embodiments, a droplet may comprise a cell and a support. In some embodiments, the droplet can be a water-in-oil droplet. In some embodiments, the droplet can further comprise a cell lysis reagent. In some embodiments, the cell lysis reagent is a cell lysis solution. In some embodiments, the cell lysis solution is a hypotonic cell lysis solution. In some embodiments, the cell lysis reagent can comprise polyoxyethylene sorbitol ester (e.g., Tween), 2-[4-(2,4,4-trimethylpentan-2-yl)phenoxy]ethanol (e.g., Triton X-100), and/or sodium dodecyl sulphate (SDS). In some embodiments, the droplet can further comprise a buffer. In some embodiments the buffer can be sodium phosphate, tris(hydroxymethyl)aminomethane, or phosphate-buffered saline.

In some cases, a dimension of a partition (e.g., droplet) may be greater than the combined width or volume of a bead, cell, biological species of interest, reagent, and/or buffer. In some embodiments, a partition (e.g., droplet) can have a mean diameter from about 15 nm to about 5 μm. In some embodiments, a partition (e.g., droplet) can have a mean diameter from about 15 nm to about 50 nm, from about 50 nm to about 100 nm, from about 100 nm to about 250 nm, from about 250 nm to about 500 nm, from about 500 nm to about 750 nm, from about 750 nm to about 1 μm, from about 1 μm to about 3 μm, or from about 3 μm to about 5 μm. In some embodiments, a partition (e.g., droplet) can have a mean diameter of from about 100 nm to about 500 nm. In some embodiments, a partition (e.g., droplet) can have a mean diameter of from about 500 nm to about 1 μm. In some embodiments, a partition (e.g., droplet) can have a mean diameter of from about 1 μm to about 3 μm. In some embodiments, a partition (e.g., droplet) can have a mean diameter of from about 3 μm to about 5 μm.

In some embodiments, a partition (e.g., droplet) can have a mean diameter of at least about 15 nm, at least about 50 nm, at least about 100 nm, at least about 250 nm, at least about 500 nm, at least about 750 nm, at least about 1 μm, at least about 2 μm, at least about 3 μm, at least about 4 μm, or at least about 5 μm. In some embodiments, a partition (e.g., droplet) can have a mean diameter of at least about 250 nm. In some embodiments, a partition (e.g., droplet) can have a mean diameter of at least about 500 nm. In some embodiments, a partition (e.g., droplet) can have a mean diameter of at least about 1 μm. In some embodiments, a partition (e.g., droplet) can have a mean diameter of at least about 2 μm. In some embodiments, a partition (e.g., droplet) can have a mean diameter of at least about 3 μm. In some embodiments, a partition (e.g., droplet) can have a mean diameter of at least about 4 μm. In some embodiments, a partition (e.g., droplet) can have a mean diameter of at least about 5 μm.

In some embodiments, a partition (e.g., droplet) can have a mean diameter of at most about 15 nm, at most about 50 nm, at most about 100 nm, at most about 250 nm, at most about 500 nm, at most about 750 nm, at most about 1 μm, at most about 2 μm, at most about 3 μm, at most about 4 μm, or at most about 5 μm. In some embodiments, a partition (e.g., droplet) can have a mean diameter of at most about 250 nm. In some embodiments, a partition (e.g., droplet) can have a mean diameter of at most about 500 nm. In some embodiments, a partition (e.g., droplet) can have a mean diameter of at most about 1 μm. In some embodiments, a partition (e.g., droplet) can have a mean diameter of at most about 2 μm. In some embodiments, a partition (e.g., droplet) can have a mean diameter of at most about 3 μm. In some embodiments, a partition (e.g., droplet) can have a mean diameter of at most about 4 μm. In some embodiments, a partition (e.g., droplet) can have a mean diameter of at most about 5 μm.

In some embodiments, a partition (e.g., droplet) can have a mean diameter of about 15 nm, about 50 nm, about 100 nm, about 250 nm, about 500 nm, about 750 nm, about 1 μm, about 2 μm, about 3 μm, about 4 μm, or about 5 μm. In some embodiments, a partition (e.g., droplet) can have a mean diameter of about 250 nm. In some embodiments, a partition (e.g., droplet) can have a mean diameter of about 500 nm. In some embodiments, a partition (e.g., droplet) can have a mean diameter of about 1 μm. In some embodiments, a partition (e.g., droplet) can have a mean diameter of about 2 μm. In some embodiments, a partition (e.g., droplet) can have a mean diameter of about 3 μm. In some embodiments, a partition (e.g., droplet) can have a mean diameter of about 4 μm. In some embodiments, a partition (e.g., droplet) can have a mean diameter of about 5 μm.

In some embodiments, a partition (e.g., droplet) can have a mean volume of from about 0.5 pL to about 750 pL. In some embodiments, a partition (e.g., droplet) can have a mean volume of from about 0.5 pL to about 50 pL, from about 50 pL to about 100 pL, from about 100 pL to about 150 pL, from about 150 pL to about 200 pL, from about 200 pL to about 250 pL, from about 250 pL to about 300 pL, from about 300 pL to about 350 pL, from about 350 pL to about 400 pL, from about 400 pL to about 450 pL, from about 450 pL to about 500 pL, from about 500 pL to about 550 pL, from about 550 pL to about 600 pL, from about 600 pL to about 650 pL, from about 650 pL to about 700 pL, or from about 700 pL to about 750 pL. In some embodiments, a partition (e.g., droplet) can have a mean volume of from about 0.5 pL to about 250 pL. In some embodiments, a partition (e.g., droplet) can have a mean volume of from about 250 pL to about 500 pL. In some embodiments, a partition (e.g., droplet) can have a mean volume of from about 500 pL to about 750 pL.

In some embodiments, a partition (e.g., droplet) can have a mean volume of at least about 0.5 pL, at least about 50 pL, at least about 100 pL, at least about 150 pL, at least about 200 pL, at least about 250 pL, at least about 300 pL, at least about 350 pL, at least about 400 pL, at least about 450 pL, at least about 500 pL, at least about 550 pL, at least about 600 pL, at least about 650 pL, at least about 700 pL, or at least about 750 pL. In some embodiments, a partition (e.g., droplet) can have a mean volume of at least about 50 pL. In some embodiments, a partition (e.g., droplet) can have a mean volume of at least about 100 pL. In some embodiments, a partition (e.g., droplet) can have a mean volume of at least about 150 pL. In some embodiments, a partition (e.g., droplet) can have a mean volume of at least about 200 pL. In some embodiments, a partition (e.g., droplet) can have a mean volume of at least about 500 pL.

In some embodiments, a partition (e.g., droplet) can have a mean volume of at most about 0.5 pL, at most about 50 pL, at most about 100 pL, at most about 150 pL, at most about 200 pL, at most about 250 pL, at most about 300 pL, at most about 350 pL, at most about 400 pL, at most about 450 pL, at most about 500 pL, at most about 550 pL, at most about 600 pL, at most about 650 pL, at most about 700 pL, or at most about 750 pL. In some embodiments, a partition (e.g., droplet) can have a mean volume of at most about 50 pL. In some embodiments, a partition (e.g., droplet) can have a mean volume of at most about 100 pL. In some embodiments, a partition (e.g., droplet) can have a mean volume of at most about 150 pL. In some embodiments, a partition (e.g., droplet) can have a mean volume of at most about 200 pL. In some embodiments, a partition (e.g., droplet) can have a mean volume of at most about 500 pL.

In some embodiments, a partition (e.g., droplet) can have a mean volume of about 0.5 pL, about 50 pL, about 100 pL, about 150 pL, about 200 pL, about 250 pL, about 300 pL, about 350 pL, about 400 pL, about 450 pL, about 500 pL, about 550 pL, about 600 pL, about 650 pL, about 700 pL, or about 750 pL. In some embodiments, a partition (e.g., droplet) can have a mean volume of about 50 pL. In some embodiments, a partition (e.g., droplet) can have a mean volume of about 100 pL. In some embodiments, a partition (e.g., droplet) can have a mean volume of about 150 pL. In some embodiments, a partition (e.g., droplet) can have a mean volume of about 200 pL. In some embodiments, a partition (e.g., droplet) can have a mean volume of about 500 pL.

### Biomolecules

In some cases, a cell is sorted within a fluidic device prior to partitioning or isolation. In some cases, a cell is sorted using a technique that separates cells by size. In some cases, a cell is sorted using a technique that separates cells by a differentiating physical property. In some cases, a sample comprising a plurality of cells can be sorted using centrifugation. In some cases, a sample comprising a plurality of cells can be sorted using fluorescence activated cell sorting (FACS). In some cases, a sample comprising a plurality of cells can be sorted using buoyancy activated cell sorting (BACS). In some cases, a sample comprising a plurality of cells can be sorted using magnetic based cell sorting. In some cases, a sample comprising a plurality of cells can be sorted using fluorescence activated cell sorting. In some cases, a sample comprising a plurality of cells can be sorted using adhesion. In some cases, a sample comprising a plurality of cells can be sorted using filtration. In some cases, a sample comprising a plurality of cells can be sorted using sedimentation. In some cases, a sample comprising a plurality of cells can be sorted using centrifugation with a density gradient medium. In some cases, a sample comprising a plurality of cells can be sorted using filtration. For example, red blood cells may be selected from a whole blood fraction prior to cell partitioning into droplets.

In some embodiments, a biomolecule is an intracellular peptide. In some embodiments, a biomolecule is an intracellular protein. In some embodiments, a biomolecule is a secreted peptide. In some embodiments, a biomolecule is a secreted protein. In some embodiments, the biomolecule is a biomolecule introduced by a post-translational modification. In some embodiments, the biomolecule is a biomolecule introduced by an enzymatic reaction. In some embodiments, the biomolecule is a biomarker indicative of a disease. In some embodiments, the biomolecule is a biomarker indicative of a health status. In some embodiments, the biomolecule can be selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), oxidized protein, and glycosylated protein. In some embodiments, the biomolecule is an oxidized protein. In some embodiments, the biomolecule is a glycosylated protein. In some embodiments, the biomolecule is HbA. In some embodiments, the biomolecule is hemoglobin A1c (HbA1c).

Tumor heterogeneity (i.e., physical state of tumor tissue containing a mixture of cancerous cells as well as healthy cells) makes protein diagnostics and therapeutic interventions challenging. Profiling the proteomes or a subset of proteome, of every individual cells provides information that identifies the number and types of different cells populating the tumor tissue. In some embodiments, a protein mutation can be associated with the presence of a cancer. In some embodiments, the mutated protein is a KRAS protein. In some embodiments, the methods of the disclosure can be used to detect the presence of a protein mutation to identify a cancer. In some embodiments, the methods of the disclosure can be used to detect the absence of a protein mutation to identify a cancer. In some embodiments, an intracellular protein target can be detected using a method of the disclosure. In some embodiments, the intracellular protein target is a kinase. In some embodiments, the kinase is an Proto-oncogene tyrosine-protein 1 (ROS1) kinase. In some embodiments, the kinase is a mitogen-activated protein kinase (MAPK).

### Capture Reagents

Various aspects of the present disclosure provide methods comprising partitioning a single cell with a capture reagent. In some embodiments, a capture reagent may comprise a bead, a polymer matrix, a nanorod, a nanodot, a protein-based nanostructure (e.g., a protein nanoparticle), a silica dot, a nucleic acid based nanostructure, a liposome, or any combination thereof. In some embodiments, a capture reagent comprises a bead. In some embodiments, a capture reagent may have a spherical shape. In some embodiments, a capture reagent may have a non-spherical shape (e.g., cubic shape). In some embodiments, a capture reagent can comprise a flat surface. In some embodiments, a capture reagent can have uneven dimensions. In some embodiments, a capture reagent can comprise ridges, grooves, dents, protrusions, or other complex surface features. In some embodiments, a capture reagent can be porous. In some embodiments, a capture reagent can be non-porous.

A capture reagent may comprise a bead (e.g., a particle). A bead may be, for example, a marble, a polymeric bead, a silica bead, a functionalized bead, a magnetic bead, an activated bead, a barcoded bead, a labeled bead, a PCA bead, a magnetic bead, or any combination thereof. In some embodiments, a bead can be a polymeric bead, for example, a polysaccharide bead, a cellulose bead, a synthetic polymer bead, or a natural polymer bead. In some embodiments, a bead can be a magnetic bead, for example, a superparamagnetic iron oxide nanoparticle (SPION).

Beads consistent with the present disclosure include micelles, liposomes, metal beads, metal oxide beads, inorganic oxide beads, iron oxide beads, silver beads, gold beads, palladium beads, platinum beads, titanium beads, silica beads, copolymer beads, terpolymer beads, polymeric beads with metal cores, polymeric beads with metal oxide cores, polystyrene sulfonate beads, polyethylene oxide beads, polyoxyethylene glycol beads, polyethylene imine beads, polylactic acid beads, polycaprolactone beads, polyglycolic acid beads, poly(lactide-co-glycolide polymer beads, cellulose ether polymer beads, polyvinylpyrrolidone beads, polyvinyl acetate beads, polyvinylpyrrolidone-vinyl acetate copolymer beads, polyvinyl alcohol beads, acrylate beads, polyacrylic acid beads, crotonic acid copolymer beads, polyethlene phosphonate beads, polyalkylene beads, carboxy vinyl polymer beads, sodium alginate beads, carrageenan beads, xanthan gum beads, gum acacia beads, Arabic gum beads, guar gum beads, pullulan beads, agar beads, chitin beads, chitosan beads, pectin beads, karaya tum beads, locust bean gum beads, maltodextrin beads, amylose beads, corn starch beads, potato starch beads, rice starch beads, tapioca starch beads, pea starch beads, sweet potato starch beads, barley starch beads, wheat starch beads, hydroxypropylated high amylose starch beads, dextrin beads, levan beads, elsinan beads, gluten beads, collagen beads, whey protein isolate beads, casein beads, milk protein beads, soy protein beads, keratin beads, polyethylene beads, polycarbonate beads, polyanhydride beads, polyhydroxyacid beads, polypropylfumerate beads, polycaprolactone beads, polyamine beads, polyacetal beads, polyether beads, polyester beads, poly(orthoester) beads, polycyanoacrylate beads, polyurethane beads, polyphosphazene beads, polyacrylate beads, polymethacrylate beads, polycyanoacrylate beads, polyurea beads, polyamine beads, polystyrene beads, poly(lysine) beads, chitosan beads, dextran beads, poly(acrylamide) beads, derivatized poly(acrylamide) beads, gelatin beads, starch beads, chitosan beads, dextran beads, gelatin beads, starch beads, poly-β-amino-ester beads, poly(amido amine) beads, poly lactic-co-glycolic acid beads, polyanhydride beads, bioreducible polymer beads, and/or 2-(3-aminopropylamino)ethanol beads, or any combination thereof.

In some cases, a capture reagent (e.g., a support or a bead) may comprise a plurality of reactive sites, which may be configured to couple to capture moieties configured to capture biomolecules. In some embodiments, a capture reagent can be functionalized with a capture moiety via covalent interactions. In some embodiments, a capture reagent can be functionalized with a capture moiety via non-covalent interactions. In some embodiments, a capture reagent can be functionalized with a reactive moiety, and the reactive moiety can bind to a reactive site of a capture moiety. In some embodiments, a capture reagent (e.g., a support or a bead) can be a streptavidin-functionalized capture reagent, and a capture moiety may be coupled to the streptavidin group of the streptavidin-functionalized capture reagent. In some embodiments, the capture moiety comprises a biotin group to couple to the streptavidin-functionalized capture reagent, and the capture moiety can further comprise a second reactive moiety to conjugate to a biomolecule. In some embodiments, the second reactive moiety can comprise a 2-pyridinylcarboxaldehyde (PCA) group. In some embodiments, the second reactive moiety can comprise a maleimide group. In some embodiments, the second reactive moiety can comprise a pyridyl disulfide group.

In some embodiments, a capture reagent can covalently bind to a capture moiety to form a conjugate. In some embodiments, the capture reagent and capture moiety are bound by a disulfide bond. In some embodiments, the capture reagent and capture moiety are bound by a thioether bond. In some embodiments, the covalent bond is formed between a first reactive moiety of a biomolecule and a second reactive moiety on a support. In some embodiments, the first reactive moiety comprises a sulfhydryl group. In some embodiments, the first reactive moiety comprises a cysteine side chain. In some embodiments, the first reactive moiety comprises a histidyl side chain. In some embodiments, the second reactive moiety comprises a pyridine carboxyaldehyde. In some embodiments, the second reactive moiety comprises a maleimide group. In some embodiments, the second reactive moiety comprises a haloacetyl group. In some embodiments, the second reactive moiety comprises a pyridyl disulfide. In some embodiments, the support comprises a plurality of second reactive moieties. In some embodiments, the covalent bond is a disulfide bond. In some embodiments, the covalent bond is a thioether bond.

In some embodiments, the methods of the disclosure can use a streptavidin-conjugated bead. In some embodiments, the methods of the disclosure can use a streptavidin-conjugated magnetic bead. In some embodiments, the methods of the disclosure can use a functionalized bead. In some embodiments, a bead can be functionalized to comprise a PCA reactive moiety. In some embodiments, a bead can be functionalized to comprise a maleimide group. In some embodiments, a bead can be functionalized to comprise a pyridyl disulfide group. In some embodiments, a streptavidin bead can be functionalized using a linker comprising a biotin moiety. In some embodiments, a streptavidin bead can be functionalized using a bifunctional linker comprising biotin as a first reactive moiety, and a second reactive moiety. In some embodiments, the second reactive moiety can comprise a PCA group, a maleimide group, or a pyridyl disulfide group.

A capture reagent (e.g., a support or bead) can have a mean diameter of from about 1 μm to about 50 μm. A capture reagent (e.g., a support or bead) can have a mean diameter of from about 15 nm to about 5 μm. A capture reagent (e.g., a support or bead) can have a mean diameter of from about 1 μm to about 5 μm. In some embodiments, a capture reagent can have a mean diameter of from about 1 μm to about 10 μm, from about 10 μm to about 20 μm, from about 20 μm to about 30 μm, from about 30 μm to about 40 μm, or from about 40 μm to about 50 μm. In some embodiments, a capture reagent can have a mean diameter of from about 10 μm to about 20 μm. In some embodiments, a capture reagent can have a mean diameter of from about 20 μm to about 30 μm. In some embodiments, a capture reagent can have a mean diameter of from about 30 μm to about 40 μm.

In some embodiments, a capture reagent can have a mean diameter of about 1 μm, about 5 μm, about 10 μm, about 15 μm, about 20 μm, about 25 μm, about 30 μm, about 35 μm, about 40 μm, about 45 μm, or about 50 μm. In some embodiments, a capture reagent can have a mean diameter of about 10 μm. In some embodiments, a capture reagent can have a mean diameter of about 20 μm. In some embodiments, a capture reagent can have a mean diameter of about 30 μm. In some embodiments, a capture reagent can have a mean diameter of about 40 μm.

A capture reagent may comprise from about 10² to about 10³, from about 10³ to about 10⁴, from about 10⁴ to about 10⁵, from about 10⁵ to about 10⁶, from about 10⁶ to about 10⁷, or from about 10⁷ to about 10⁸ reactive sites. A capture reagent may comprise at least about 10², at least about 10³, at least about 10⁴, at least about 10⁵, at least about 10⁶, at least about 10⁷, or at least about 10⁸ reactive sites. A capture reagent may comprise about 10², about 10³, about 10⁴, about 10⁵, about 10⁶, about 10⁷, or about 10⁸ reactive sites. A capture reagent may comprise at most about 10⁸, at most about 10⁷, at most about 10⁶, at most about 10⁵, at most about 10⁴, at most about 10³, or at most about 10² reactive sites.

In some embodiments, a capture reagent may comprise a single type of reactive site. In some embodiments, a capture reagent may comprise multiple types of reactive sites. In some embodiments, the reactive sites may be evenly distributed across the surface of the capture reagent. In some embodiments, the reactive sites may be unevenly distributed across the surface of the capture reagent. In some embodiments, the reactive sites may be clustered by type across the surface of the capture reagent. In some embodiments, the reactive sites may be randomly distributed across the surface of the capture reagent. In some embodiments, the reactive sites may be spaced so as to minimize denaturation of captured proteins. For example, reactive sites comprising biomolecule-specific capture moieties may comprise an average separation of at least 5 nm, thereby minimizing crowding of the biomolecule (which comprises a dimension of about 5 nm) and/or possible denaturation of the biomolecule bound to a capture reagent.

In some embodiments, reactive sites may comprise an average spacing of from about 1 nm to about 2 nm, from about 2 nm to about 4 nm, from about 4 nm to about 6 nm, from about 6 nm to about 8 nm, from about 8 nm to about 10 nm, from about 10 nm to about 12 nm, from about 12 nm to about 14 nm, from about 14 nm to about 16 nm, from about 16 nm to about 18 nm, or from about 18 nm to about 20 nm. In some embodiments, reactive sites may comprise an average spacing of at least about 1 nm, at least about 1.5 nm, at least about 2 nm, at least about 2.5 nm, at least about 3 nm, at least about 4 nm, at least about 5 nm, at least about 6 nm, at least about 8 nm, at least about 10 nm, at least about 12 nm, at least about 15 nm, or at least about 20 nm. In some embodiments, reactive sites may comprise an average spacing of at least about 1 nm. In some embodiments, reactive sites may comprise an average spacing of at least about 2 nm. In some embodiments, reactive sites may comprise an average spacing of at least about 5 nm. In some embodiments, reactive sites may comprise an average spacing of at least about 10 nm.

In some embodiments, reactive sites may comprise an average spacing of at most about 1 nm, at most about 1.5 nm, at most about 2 nm, at most about 2.5 nm, at most about 3 nm, at most about 4 nm, at most about 5 nm, at most about 6 nm, at most about 8 nm, at most about 10 nm, at most about 12 nm, at most about 15 nm, or at most about 20 nm. In some embodiments, reactive sites may comprise an average spacing of at most about 1 nm. In some embodiments, reactive sites may comprise an average spacing of at most about 2 nm. In some embodiments, reactive sites may comprise an average spacing of at most about 5 nm. In some embodiments, reactive sites may comprise an average spacing of at most about 10 nm.

In some embodiments, reactive sites may comprise an average spacing of about 1 nm, about 1.5 nm, about 2 nm, about 2.5 nm, about 3 nm, about 4 nm, about 5 nm, about 6 nm, about 8 nm, about 10 nm, about 12 nm, about 15 nm, or about 20 nm. In some embodiments, reactive sites may comprise an average spacing of about 1 nm. In some embodiments, reactive sites may comprise an average spacing of about 2 nm. In some embodiments, reactive sites may comprise an average spacing of about 5 nm. In some embodiments, reactive sites may comprise an average spacing of about 10 nm.

A capture reagent (e.g., a bead) may comprise a plurality of capture moieties. A plurality of capture moieties may be coupled to a plurality of reactive sites. Conversely, a plurality of capture moieties may be directly attached to the capture reagent. In some cases, a capture moiety comprises a chemically reactive group that is configured to couple to specific type of biomolecule. In some cases, a capture moiety comprises a chemically reactive group that forms a thioether bond. In some cases, a capture moiety comprises a chemically reactive group that forms a disulfide bond. In some embodiments, the chemically reactive group comprises a maleimide group. In some embodiments, the chemically reactive group comprises a PCA group. In some embodiments, the chemically reactive group comprises a haloacetyl group. In some embodiments, the chemically reactive group comprises a pyridyl disulfide group. In some embodiments, the chemically reactive group is reactive toward the N-terminus of a peptide. In some embodiments, the chemically reactive group is reactive toward a specific amino acid of a peptide. In some embodiments, the chemically reactive group is reactive toward cysteine residues. In some cases, a capture moiety from among the plurality of capture moieties may comprise an affinity for a particular biomolecule or class of biomolecules. In some cases, a support or bead can comprise a plurality of capture moieties.

A capture reagent may comprise from about 10² to about 10³, from about 10³ to about 10⁴, from about 10⁴ to about 10⁵, from about 10⁵ to about 10⁶, from about 10⁶ to about 10⁷, or from about 10⁷ to about 10⁸ capture moieties. A capture reagent may comprise at least about 10², at least about 10³, at least about 10⁴, at least about 10⁵, at least about 10⁶, at least about 10⁷, or at least about 10⁸ capture moieties. A capture reagent may comprise about 10², about 10³, about 10⁴, about 10⁵, about 10⁶, about 10⁷, or about 10⁸ capture moieties. A capture reagent may comprise at most about 10⁸, at most about 10⁷, at most about 10⁶, at most about 10⁵, at most about 10⁴, at most about 10³, or at most about 10² capture moieties. In some cases, a capture moiety comprises an aromatic or heteroaromatic carboxaldehyde. In some cases, the aromatic or heteroaromatic carboxaldehyde is PCA. In some cases, the plurality of capture moieties comprises an aldehyde, a maleimide, a thiol, a carboxylic acid, a succinimide, a carbonate, a carbamate, a urea, an amidoester, an azide, an acryl azide, an aryl dihalides, a sulfone, a glutaraldehyde, a dimaleimide, an anhydride, a dicarboxylate, an N-hydroxysuccimide ester, tetrazine, alkene, alkyne, maleimide or any combination thereof.

In some cases, capture moiety binding to a bead or a reactive site on a bead is irreversible. In some embodiments, a capture moiety is covalently bound to a peptide of a biomolecule. In some embodiments, a capture moiety is covalently bound to an amino acid residue of a biomolecule. In some embodiments, a capture moiety is covalently bound to an amino acid residue of a biomolecule by a disulfide bond. In some embodiments, a capture moiety is covalently bound to an amino acid residue of a biomolecule by a thioether bond.

In some cases, capture moiety binding to a bead or reactive site on a bead is reversible. For example, a biotin-containing capture moiety may be eluted from a bead comprising streptavidin reactive sites by addition of formic acid in acetonitrile. A capture moiety may also comprise a cleavable moiety, such as a hydrolysable (2-pyridyldithio)pentanoate (SPP) linker or diazobenzene moiety.

In some cases, a capture moiety comprises an epitope specific antibody. In some cases, the epitope specific antibody is configured to bind a biomolecule of a cell. In some cases, the epitope specific antibody is configured to bind to a reactive group of a capture moiety, such as a bi-functional capture moiety with two identical or different reactive groups. For example, a reactive site may comprise streptavidin coupled to a bi-functional linker comprising a streptavidin binding moiety (e.g., biotin) and a peptide capture moiety (e.g., maleimide). In some cases, the bi-functional linker comprises biotin-PEG(11)-maleimide, biotin-PEG(11)-PCA, biotin-PEG(11)-pyridyl disulfide, biotin-PEG(11)-haloacetyl.

In some cases, a capture moiety is configured for cleavage. A capture moiety may comprise a cleavable domain. A capture moiety may also be coupled to a linker (e.g., a bi-functional linker) which comprises a cleavable domain. The cleavable domain may be a chemically cleavable domain, a pH-cleavable domain, a photocleavable domain, an enzymatically cleavable domain, or any combination thereof. In some cases, a capture moiety may be cleaved by a reversing agent (e.g., hydrazine).

In some cases, a bead may comprise a plurality of reactive sites or capture moieties configured to capture a specific percentage of proteins or peptides from a cell. For example, a partition (e.g., droplet) may comprise a single cell (e.g., a red blood cell) that contains about 5×10⁹ proteins and a bead comprising 5×10⁵ peptide-specific reactive sites, and thus be configured to capture 0.01% of the peptides or proteins derived from the single cell. In some embodiments, a partition may comprise a single cell (e.g., a small prokaryotic cell) that contains 10⁶ proteins or peptides and a bead comprising 3×10⁶ peptide specific reactive sites (e.g., capture moieties), and thus be configured to capture all of the proteins or peptides derived from the single cell.

In some embodiments, a support or bead may comprise a plurality of reactive sites that are configured to capture from about 0.001% to about 0.01%, from about 0.01% to about 0.1%, from about 0.1% to about 1%, from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of the peptides or proteins derived from a single cell. In some embodiments, a support or bead may comprise a plurality of reactive sites that are configured to capture at least about 0.001%, at least about 0.01%, at least about 0.1%, at least about 1%, at least about 10%, at least about 20%, at least about 30%, at least about 40%, at least about 50%, at least about 60%, at least about 70%, at least about 80%, at least about 90%, or about 100% of the peptides or proteins derived from a single cell. In some embodiments, a support or bead may comprise a plurality of reactive sites that are configured to capture about 0.001%, about 0.01%, about 0.1%, about 1%, about 10%, about 20%, about 30%, about 40%, about 50%, about 60%, about 70%, about 80%, about 90%, or about 100% of the peptides or proteins derived from a single cell. In some embodiments, a support or bead may comprise a plurality of reactive sites that are configured to capture at most about 0.001%, at most about 0.01%, at most about 0.1%, at most about 1%, at most about 10%, at most about 20%, at most about 30%, at most about 40%, at most about 50%, at most about 60%, at most about 70%, at most about 80%, at most about 90%, or about 100% of the peptides or proteins derived from a single cell.

### Barcode Molecules

In some cases, a partition (e.g., droplet) may comprise a barcode molecule or a plurality of barcode molecules. The barcode molecule may be attached (e.g., covalently bound) to a bead, such as to a reactive site on the bead. A barcode may be attached to or disposed within a capture moiety. For example, a capture moiety may comprise a biotin moiety enabling attachment to a bead-bound streptavidin, a maleimide moiety for binding a peptide N-terminus, and/or an oligopeptide barcode molecule that uniquely identifies a cell, bead, or partition. A barcode molecule may be attached to a molecule derived from a cell. For example, a cell may be lysed within a partition, and the N-terminal of a peptide from the cell may be coupled to a maleimide moiety on a barcode molecule. A barcode molecule may be cleavable from a bead or capture moiety. For example, a barcode molecule may comprise a hydrolysable disulfide group.

In some cases, a barcode molecule comprises a sequence that uniquely identifies a droplet, a capture reagent (e.g., a bead), or cell. As such, a barcode may enable cell-of-origin identification of analytes from a pooled sample. A plurality of partitions may comprise a plurality of distinct barcodes, such that a biomolecule or a capture reagent may later be identified from a pooled sample.

A barcode molecule may comprise a deoxyribonucleic acid (DNA). A barcode molecule may comprise a ribonucleic acid (RNA). A barcode molecule may comprise a peptide nucleic acid. A barcode molecule may comprise a peptide (e.g., an oligopeptide, a polypeptide, or a protein). In some cases, a capture moiety comprises a barcode molecule. In some cases, a bi-functional linker comprises a barcode molecule. In some cases, a barcode remains attached to a capture moiety or bi-functional linker following cleavage or dissociation from a capture reagent, such as a bead.

In some cases, a barcode molecule comprises an oligopeptide comprising from about 2 to 50 amino acids. In some embodiments, a barcode molecule can comprise from about 1 to about 10 amino acids, from about 10 to about 20 amino acids, from about 20 to about 30 amino acids, from about 30 to about 40 amino acids, or from about 40 to about 50 amino acids. In some cases, the barcode molecule may comprise glutamic acid, aspartic acid, cysteine, methionine, lysine, arginine, tyrosine, tryptophan, and/or phenylalanine. In some cases, the barcode may comprise a modified amino acid, such as a methylimidazole or phosphotyrosine. In some cases, the barcode may comprises a non-natural amino acid. In some cases, an oligopeptide barcode may be identified by sequencing. In some cases, an oligopeptide barcode may be identified by fluorosequencing. In some cases, an oligopeptide barcode can be identified with tandem mass spectrometry.

In some cases, a method comprises detecting a barcode molecule coupled to a biomolecule. In some cases, the barcode molecule may be cleaved from the biomolecule. In some cases, detecting a barcode molecule comprises mass spectrometric analysis (e.g., tandem mass spectrometry). In some cases, detecting a barcode molecule comprises fluorosequencing. In some cases, detecting a barcode molecule comprises optical detection. In some cases, the optical detection comprises imaging. In some cases, the detecting comprises fluorosequencing at least a portion of the barcode molecule. In some cases, each barcode molecule from among a plurality of barcode molecules utilized in an assay has a unique mass spectrometric or tandem mass spectrometric fingerprint. In some cases, each bead from among the plurality of beads utilized in an assay is associated with a unique type of barcode molecule.

In some cases, detecting a barcode molecule comprises sequencing, such as nanopore sequencing, nucleic acid next generation sequencing (NGS), or peptide fluorosequencing. In some cases, the sequencing comprises Edman degradation. In some cases, detecting the barcode molecule identifies the cell that a biomolecule originated from. In some cases, this principle may be extended to multi-cell assays, wherein the cell of origin may be assigned for each of a plurality of biomolecules.

Methods and/or Systems for Assaying a Peptide or Proteins

The present disclosure provides methods and/or systems for analyzing a biomolecule from a single cell. In some cases, the methods involve partitioning a single cell and detecting at least one biomolecule derived from the cell. In some embodiments, a partition is a water-in-oil droplet. In some embodiments, exactly one cell and one bead can be encapsulated in a water-in-oil droplet. In some cases, the partition comprises one cell and two or more beads. In some cases, the partition comprises two or more cells and one bead. In some cases, the partition comprises two or more cells and two or more beads. In some cases, the bead comprises a plurality of reactive sites configured to capture biomolecules (e.g., peptides) from the single cell. In some cases, the plurality of reactive sites comprises a capture moiety. In some cases, a method comprises rendering the contents of the single cell accessible (e.g., lysing the cell) within the partition. In some cases, a method comprises rendering the contents of a nucleus of a cell accessible within the partition. In some cases, the methods comprise immobilizing a plurality of biomolecules derived from the single cell onto the bead, for example by coupling (e.g., chemically coupling) a biomolecule from the cell with a capture moiety. In some cases, the immobilization is reversible (e.g., by capture moiety cleavage). In some cases, the methods comprise coupling a barcode molecule to a biomolecule or a bead.

In some cases, the cell may be derived from a blood sample. In some cases, the cell may be derived from a tissue sample. In some cases, the cell may be derived from a cell culture. In some cases, the cell may be isolated from extracellular species prior to assaying. For example, obtaining red blood cells from a sample may comprise first removing extracellular components (e.g., cell-free DNA and/or albumin) from the sample, purified on a first type of bead, and then partitioned with a second type of bead for the assaying.

In some embodiments, the cell is a red blood cell, a stem cell, a bone cell, a muscle cell, fat cell, skin cell, nerve cell, endothelial cell, sex cell, pancreatic cell, or cancer cell. In some embodiments, the cell is a red blood cell. In some embodiments, the cell is a skin cell. In some embodiments, the cell is a muscle cell. In some embodiments, the cell is a endothelial cell. In some embodiments, the cell is a cancer cell.

In some embodiments, a cell in a droplet is lysed, rendering the contents of the cell accessible. In some embodiments, a droplet can comprise an aqueous solution. In some cases, the method comprises liberating or collecting the biomolecule from a cell, an exosome, an organelle, a nucleus, a vesicle, or any combination thereof. In some embodiments, a droplet comprises an aqueous solution. In some embodiments, a droplet can comprise a cell lysis solution. In some embodiments, a droplet can comprise a hypotonic lysis solution. In some embodiments, the cell lysis solution comprises a polyoxyethylene sorbitol ester. In some embodiments, the cell lysis solution comprises 2-[4-(2,4,4-trimethylpentan-2-yl)phenoxy]ethanol. In some embodiments, the cell lysis solution comprises sodium dodecyl sulphate. In some embodiments, the cell lysis solution comprises trifluoroethanol. In some embodiments, the lysis may comprise heating, freezing, sonication, cytolysis, chemical lysis, osmotic shock, cavitation, chemicals (e.g., detergents), proteins, alkaline or acidic conditions, electrical current, or any combination thereof. The lysis may be a cell-type specific form of lysis. In some embodiments, a lysis method or set of lysis reagents may affect red blood cells but leave other types of cells unaffected. In some embodiments, the lysis may also comprise conditions that stabilize a component of the cell lysate. In some embodiments, a red blood cell lysis buffer may provide a stable medium for hemoglobin. A lysis method or set of lysis reagents may be optimized for fast or slow lysis. In some embodiments, a cell lysis solution or cell lysis buffer may perform lysis on the order of nanoseconds, microseconds, milliseconds, seconds, minutes, or hours.

In some embodiments, a droplet can comprise an aqueous solution. In some embodiments, a droplet can further comprise a buffer. In some embodiments, the buffer comprises sodium phosphate. In some embodiments, the buffer comprises tris(hydroxymethyl)aminomethane. In some embodiments, the buffer comprises phosphate-buffered saline.

In some cases, rendering the contents of the cell accessible may comprise permeabilizing the cell. The permeabilizing may comprise heating, electrical current, osmotic shock, chemicals (e.g., detergents), or any combination thereof. The permeabilization may result in the majority of intracellular components or as little as 0.1% of intracellular components intracellular components diffusing from a cell. In some cases, the permeabilization leaves an organelle intact. In some cases, the permeabilization is reversible.

The methods disclosed herein further comprise contacting at least one biomolecule from a cell and a support (e.g., bead) in a droplet. In some embodiments, the biomolecule is an intracellular peptide. In some embodiments, the biomolecule is an intracellular protein. In some embodiments, the biomolecule is a secreted peptide. In some embodiments, the biomolecule is a secreted protein. In some embodiments, the contacting forms a conjugate comprising a support (e.g., bead) and/or at least one biomolecule. In some embodiments, the contacting results in a reactive group of the biomolecule and/or a capture moiety of the support or bead forming a bond. In some embodiments, the bond is a covalent bond. In some embodiments, the bond is a covalent bond between the support or bead and a reactive group of a capture moiety. In some embodiments, the bond is a covalent bond between the support or bead and a reactive group of a biomolecule. In some embodiments, the bond is a disulfide bond. In some embodiments, the bond is a thioether bond.

In some cases, the method comprises capturing a plurality of biomolecules on the bead, such as a plurality of peptides or proteins from the cell. The bead may comprise a plurality of reactive sites configured to capture at least a portion of the that is configured to capture at least 0.001%, at least 0.005%, at least 0.01%, at least 0.02%, at least 0.03%, at least 0.05%, at least 0.08%, at least 0.1%, at least 0.12%, at least 0.15%, at least 0.2%, at least 0.3%, at least 0.4%, at least 0.5%, at least 0.6%, at least 0.8%, at least 1%, at least 1.2%, at least 1.5%, at least 2%, at least 2.5%, at least 3%, at least 4%, or at least 5% of the plurality of biomolecules (e.g., peptides or proteins) derived from the cell. The biomolecules may be a particular type or class of biomolecules. For example, a bead may capture 0.1% of all proteins from a cell, or a bead may capture 0.1% of all proteins of a specific class or type (e.g., hemoglobin) from a cell.

In some cases, the plurality of cells may be incubated with a plurality of beads. The ratio of the number of cells from the plurality of cells to the number of beads from the plurality of beads may be from about 100:1 to about 80:1, from about 80:1 to about 60:1, from about 60:1 to about 50:1, from about 50:1 to about 40:1, from about 40:1 to about 30:1, from about 30:1 to about 20:1, from about 20:1 to about 15:1, from about 15:1 to about 10:1, from about 10:1 to about 8:1, from about 8:1 to about 5:1, from about 5:1 to about 2:1, from about 2:1 to about 1.5:1, from about 1.5:1 to about 1:1, from about 1:1 to about 1:1.5, from about 1:1.5 to about 1:2, from about 1:2 to about 1:5, from about 1:5 to about 1:8, from about 1:8 to about 1:10, from about 1:10 to about 1:15, from about 1:15 to about 1:20, from about 1:20 to about 1:30, from about 1:30 to about 1:40, from about 1:40 to about 1:50, from about 1:50 to about 1:60, from about 1:60 to about 1:80, or from about 1:80 to about 1:100. In some cases, the plurality of cells may be incubated with a plurality of beads. The ratio of the number of cells from the plurality of cells to the number of beads from the plurality of beads may be at least about 100:1, at least about 80:1, at least about 60:1, at least about 50:1, at least about 40:1, at least about 30:1, at least about 20:1, at least about 15:1, at least about 10:1, at least about 8:1, at least about 5:1, at least about 2:1, at least about 1.5:1, at least about 1:1, at least about 1:1.5, at least about 1:2, at least about 1:5, at least about 1:8, at least about 1:10, at least about 1:15, at least about 20:1, at least about 30:1, at least about 40:1, at least about 50:1, at least about 60:1, at least about 80:1, or at least about 1:100. The ratio of the number of cells from the plurality of cells to the number of beads from the plurality of beads may be about 100:1, about 80:1, about 60:1, about 50:1, about 40:1, about 30:1, about 20:1, about 15:1, about 10:1, about 8:1, about 5:1, about 2:1, about 1.5:1, about 1:1, about 1:1.5, about 1:2, about 1:5, about 1:8, about 1:10, about 1:15, about 20:1, about 30:1, about 40:1, about 50:1, about 60:1, about 80:1, or about 1:100. The ratio of the number of cells from the plurality of cells to the number of beads from the plurality of beads may be at most about 100:1, at most about 80:1, at most about 60:1, at most about 50:1, at most about 40:1, at most about 30:1, at most about 20:1, at most about 15:1, at most about 10:1, at most about 8:1, at most about 5:1, at most about 2:1, at most about 1.5:1, at most about 1:1, at most about 1:1.5, at most about 1:2, at most about 1:5, at most about 1:8, at most about 1:10, at most about 1:15, at most about 20:1, at most about 30:1, at most about 40:1, at most about 50:1, at most about 60:1, at most about 80:1, or at most about 1:100.

A droplet may exist for a predetermined length of time prior to being opened. A droplet may exist for from about 1 minute to about 2 minutes, from about 2 minutes to about 3 minutes, from about 3 minutes to about 5 minutes, from about 5 minutes to about 8 minutes, from about 8 minutes to about 12 minutes, from about 12 minutes to about 15 minutes, from about 15 minutes to about 20 minutes, from about 20 minutes to about 30 minutes, from about 30 minutes to about 40 minutes, from about 40 minutes to about 50 minutes, from about 50 minutes to about 60 minutes, from about 60 minutes to about 80 minutes, from about 80 minutes to about 100 minutes, from about 100 minutes to about 120 minutes, from about 120 minutes to about 150 minutes, from about 150 minutes to about 180 minutes, from about 180 minutes to about 210 minutes, from about 210 minutes to about 240 minutes, from about 240 minutes to about 300 minutes, from about 300 minutes to about 360 minutes, from about 360 minutes to about 540 minutes, from about 540 minutes to about 720 minutes, from about 720 minutes to about 900 minutes, from about 900 minutes to about 1080 minutes, from about 1080 minutes to about 1440 minutes, from about 1440 minutes to about 2880 minutes, or from about 2880 minutes to about 4320 minutes. A droplet may exist for a predetermined length of time prior to being opened. A droplet may exist for at least about 1 minute, at least about 2 minutes, at least about 3 minutes, at least about 5 minutes, at least about 8 minutes, at least about 12 minutes, at least about 15 minutes, at least about 20 minutes, at least about 30 minutes, at least about 40 minutes, at least about 50 minutes, at least about 60 minutes, at least about 80 minutes, at least about 100 minutes, at least about 120 minutes, at least about 150 minutes, at least about 180 minutes, at least about 210 minutes, at least about 240 minutes, at least about 300 minutes, at least about 360 minutes, at least about 540 minutes, at least about 720 minutes, at least about 900 minutes, at least about 1080 minutes, at least about 1440 minutes, at least about 2880 minutes, or at least about 4320 minutes. A droplet may exist for about 1 minute, about 2 minutes, about 3 minutes, about 5 minutes, about 8 minutes, about 12 minutes, about 15 minutes, about 20 minutes, about 30 minutes, about 40 minutes, about 50 minutes, about 60 minutes, about 80 minutes, about 100 minutes, about 120 minutes, about 150 minutes, about 180 minutes, about 210 minutes, about 240 minutes, about 300 minutes, about 360 minutes, about 540 minutes, about 720 minutes, about 900 minutes, about 1080 minutes, about 1440 minutes, about 2880 minutes, or about 4320 minutes. A droplet may exist for at most about 1 minute, at most about 2 minutes, at most about 3 minutes, at most about 5 minutes, at most about 8 minutes, at most about 12 minutes, at most about 15 minutes, at most about 20 minutes, at most about 30 minutes, at most about 40 minutes, at most about 50 minutes, at most about 60 minutes, at most about 80 minutes, at most about 100 minutes, at most about 120 minutes, at most about 150 minutes, at most about 180 minutes, at most about 210 minutes, at most about 240 minutes, at most about 300 minutes, at most about 360 minutes, at most about 540 minutes, at most about 720 minutes, at most about 900 minutes, at most about 1080 minutes, at most about 1440 minutes, at most about 2880 minutes, or at most about 4320 minutes.

A droplet may exist for a predetermined length of time prior to being opened. A droplet may exist for at most about 1 minute, at most about 2 minutes, at most about 3 minutes, at most about 5 minutes, at most about 8 minutes, at most about 12 minutes, at most about 15 minutes, at most about 20 minutes, at most about 30 minutes, at most about 40 minutes, at most about 50 minutes, at most about 60 minutes, at most about 80 minutes, at most about 100 minutes, at most about 120 minutes, at most about 150 minutes, at most about 180 minutes, at most about 210 minutes, at most about 240 minutes, at most about 300 minutes, at most about 360 minutes, at most about 540 minutes, at most about 720 minutes, at most about 900 minutes, at most about 1080 minutes, at most about 1440 minutes, at most about 2880 minutes, or at most about 4320 minutes.

A plurality of droplets may be collected (e.g., within a compartment of a fluidic device) and/or stably incubated during a process (e.g., a coupling reaction between a peptide capture agent on a bead and/or a cell derived peptide). The length of time which a droplet exists may be sufficient for one or more processes (e.g., cellular lysis and/or protein collection on a bead) to occur and optionally to approach completion.

The methods disclosed herein further comprise collecting a biomolecule conjugate into a sample. After biomolecule conjugates are formed, a droplet may be broken. In some embodiments, contents of a droplet comprising at least one biomolecule and a support can be released by adding a reagent. In some embodiments, the reagent is an organic solvent, for example, isopropanol.

After the contents of a droplet are released, the contents of the droplet can be collected into a separate partition or a separate sample. In some embodiments, the sample is a well. In some embodiments, the sample is a tube. In some embodiments, the contents of a plurality of droplets may be combined within a sample (e.g., well or tube). For example, a fluidic device may collect droplets in a partition (e.g., a test tube).

A droplet may be collected as a separate partition. In some cases, a droplet may be merged with a separate partition. For example, a droplet may be collected in a well or a tube. Two or more droplets may be combined (e.g., at the confluence of two or more channels) to form a combined partition in a well or tube, and/or the well or tube may comprise the entirety or a subset of the contents of the two or more combined droplets.

A partition may comprise a container, for example, a well or a tube. In some cases, partitioning into a container comprises fluid transfer (e.g., pipetting) into the container. In some cases, partitioning into a container comprises gravimetric settling. In some cases, partitioning into a container comprises centrifugation. In some embodiments, partitioning comprises applying a magnetic field to a support, wherein the support is a magnetic bead. In some cases, a droplet is deposited into a well. In some cases, a droplet is deposited into a well by directing an outlet stream of a system or device toward a well. In some cases, partitioning may comprise partitioning a plurality of cells into a plurality of wells.

The chemical modifications may be performed prior to cellular partitioning, within a partition, or after a biomolecule has been removed from a partition. In some cases, a method comprises chemically modifying a biomolecule with at least one detectable moiety to produce at least one labeled biomolecule, after the collecting. In some cases, a method comprises chemically modifying a biomolecule prior to detecting the biomolecule. In some cases, a method comprises chemically modifying a biomolecule after the partitioning but prior to detecting the biomolecule.

In some cases, the chemical modification comprises reducing the biomolecule. In some cases, the reducing agent comprises 2-mercaptoethanol, 2-mercaptoethanolamine, TCEP, (tris(2-carboxyethyl)phosphine, dithiothreitol, ascorbic acid, cysteine hydrochloride, sulfite (e.g., sodium sulfite), or thioglycolate (e.g., sodium thioglycolate). In some cases, a protein may be reduced prior to digestion. In some cases, a protein may be reduced prior to analysis. For example, a disulfide bond or a cofactor may be reduced (e.g., hemoglobin ferric heme may be reduced to ferrous heme).

In some cases, chemical modification comprises fragmenting a biomolecule. In some cases, the fragmenting comprises digestion (e.g., enzymatic digestion). In some cases, the digestion comprises enzymatic peptide digestion, for example by a protease such as pepsin, aminopeptidase, gelatinase B, gelatinase A, alpha-chymotrypsin, carboxypeptidase, endoproteinase, trypsin, chymotrypsin, lumen pepsin, elastase, dipeptidyl peptidase, enteropeptidase and/or hydrolase, NS3, granzyme B, thrombin, plasmin, urokinase, sodium thiosulfite (Na₂S₂O₄) or any combination thereof. In some cases, the digestion comprises a chemical peptide digestion, for example by cyanogen bromide, BNPS skatole, formic acid, hydroxylamine, 2-nitro-5-thiocyanobenzoic acid (NTCB), or any combination thereof. For example, hemoglobin liberated via permeabilization of a red blood cell may be fragmented by a protease prior to analysis.

The method may further comprise chemically modifying the biomolecule prior to detection. The chemical modifying may comprise reducing the biomolecule (e.g., reducing a disulfide bond in the biomolecule). The chemical modifying may comprise digesting the biomolecule (e.g., cleaving the biomolecule with a protease). The chemical modifying may comprise denaturing the biomolecule (e.g., denaturing a peptide). The denaturing may comprise formamide, sodium salicylate, dimethyl sulfoxide (DMSO), propylene glycol, perchlorate, sodium dodecyl sulfate, guanidine, urea, or any combination thereof.

In some cases, the assaying comprises coupling an antibody to the biomolecule. The antibody may comprise a paratope for a particular biomolecule, a class of biomolecules (e.g., a paratope for a family of proteins), a chemically modified version of a biomolecule (e.g., an oxidized form of a biomolecule or a protein with a particular post-translational modification). In some cases, the post-translational modification comprises glycation. In some cases, the post-translational modification comprises glycosylation. In some cases, the post-translational modification comprises oxidation.

In some cases, the antibody comprises a label, and the assaying comprises detecting the label. In some cases, each type of antibody from among a plurality of antibodies may comprise a unique type of label. A label may be optically detectable (e.g., the label may comprise a fluorophore). In some cases, the detecting of the label comprises electrochemical detection, optical detection, electrophoretic detection, imaging (e.g., electron microscopy), sequencing, antibodies (e.g., immunostaining), or any combination thereof. In some embodiments, the labeling comprises immunostaining.

In some cases, assaying a biomolecule comprises coupling an amino acid specific label to the biomolecule. In some cases, the amino acid specific label comprises a detectable moiety (e.g., an optically detectable moiety). In some cases, the detectable moiety is a fluorescent moiety. In some cases, the amino acid specific label has specificity for coupling to histidine, tyrosine, lysine, arginine, cysteine, or amino acids comprising carboxylic acids in their R-groups. In some embodiments, the fluorescent moiety comprises an antibody. In some embodiments, the fluorescent moiety comprises a fluorescent dye. In some embodiments, the fluorescent moiety comprises Alexa Fluor 488. In some embodiments, the fluorescent moiety comprises Atto647N.

In some cases, the biomolecule is labeled after a conjugate comprising a support and at least one biomolecule is formed. In some cases, the biomolecule is labeled after the conjugate is collected into a container. In some embodiments, the conjugate is collected into a container, washed to remove cell lysis reagents and undesired cell material, then the biomolecule is labeled. In some embodiments, the carboxylate side chains of glutamyl/aspartyl residues may be labeled with a detectable label. In some embodiments, side chains of biomolecules can be selectively labeled, for example, to target cysteine and lysine. In some embodiments, side chains of biomolecules are selectively labeled using iodoacetamide, guanidination reagents, and tryptophan labeling reagents.

In some cases, the assaying comprises identifying the biomolecule. In some cases, the assaying comprises identifying a chemical modification on the biomolecule (e.g., post-translational modifications). In some cases, the assaying comprises determining the concentration or expression level of a biomolecule.

Various aspects of the present disclosure provide methods for analyzing a plurality of cells from a biological sample. Some aspects of the present disclosure provide methods for partitioning a plurality of cells into separate partitions. In some cases, the partitioning may result in a partition receiving a single cell.

In some cases, the partitioning may result in from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 95%, from about 95% to about 98%, from about 98% to about 99%, from about 99% to about 99.5%, from about 99.5% to about 99.9% of cells from among the plurality of cells ending up in single cell partitions. In some cases, the partitioning may result in at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.5%, at least about 99.9%, at least about 99.99%, or at least about 99.999% of cells from among the plurality of cells ending up in single cell partitions. In some cases, the partitioning may result in about 10%, about 20%, about 30%, about 40%, about 50%, about 60%, about 70%, about 80%, about 90%, about 95%, about 98%, about 99%, about 99.5%, about 99.9%, about 99.99%, or about 99.999% of cells from among the plurality of cells ending up in single cell partitions. In some cases, the partitioning may result in at most about 10%, at most about 20%, at most about 30%, at most about 40%, at most about 50%, at most about 60%, at most about 70%, at most about 80%, at most about 90%, at most about 95%, at most about 98%, at most about 99%, at most about 99.5%, at most about 99.9%, at most about 99.99%, or at most about 99.999% of cells from among the plurality of cells ending up in single cell partitions.

A method may quantitate a biomolecule, for example by determining the copy number or concentration within the cell from which the biomolecule was derived. For example, a method may comprise capturing proteins derived from a red blood cell on a bead, identifying at least a subset of the proteins on the bead, and/or extrapolating the populations of identified proteins to determine the populations of those proteins within the red blood cell. The assaying may also identify a structure (e.g., glycation at a particular site or a particular glycosylation pattern) or a portion of a structure of a biomolecule and can furthermore quantify a population of biomolecules comprising the structure or the portion of the structure thereof. For example, the assaying may identify a post-translational modification on a protein or may identify the isoforms of a particular protein present in a cell. In cases where the biomolecule is oligomeric or polymeric (e.g., when the biomolecule comprises a peptide), the assaying may identify a sequence of the biomolecule. In some cases, the assaying may identify or quantitate a plurality of biomolecules within a cell.

A method may comprise releasing a biomolecule bound to a bead or a portion of a biomolecule bound to a bead. Released biomolecules or portions thereof may be collected for further analysis. For example, a method may comprise releasing a plurality of peptides from a bead, labeling the plurality of peptides with amino acid specific labels, immobilizing the plurality of peptides to a slide, and/or subjecting the plurality of peptides to fluorosequencing. The releasing may be performed within or outside of a partition. For example, the releasing may be performed within a droplet or a well comprising contents of a droplet or may be performed on a pooled plurality of beads collected from a plurality of droplets. The releasing may comprise on-bead chemical modification or digestion of a biomolecule bound thereto.

A method may comprise digesting (e.g., trypsinization) a peptide bound to a bead, thereby liberating a peptide fragment or a plurality of peptide fragments from the peptide and the bead. In some cases, the method may comprise (i) providing a plurality of peptides coupled to a bead, (ii) denaturing the plurality of peptides to provide peptide fragments. The peptide fragments can be collected or utilized in further assays. In some cases, the method may comprise (i) providing a plurality of peptides coupled to a bead, (ii) denaturing the plurality of peptides; and (iii) reducing the covalent bonds between the plurality of peptides and the bead to provide peptide fragments. In some cases, the method may comprise (i) providing a plurality of peptides coupled to a bead, (ii) denaturing the plurality of peptides by suspending the bead in a pH 8.5 solution comprising 4 M guanidium chloride, (iii) reducing disulfide bonds of the plurality of peptides by incubating the bead in 2 μmM dithiothreitol (DTT) for 2 hours, (iv) resuspending the bead in pH 7.8 NH₄(HCO₃), and/or (v) contacting the plurality of peptides with 200 ng trypsin for 12 hours at 37° C. The plurality of peptide fragments may be collected and/or utilized in further assays, such as a fluorosequencing assay.

A method may comprise a calibrant. The calibrant may enable or increase the accuracy of target analyte quantitation. The calibrant may comprise a bead functionalized with a known amount of a target biomolecule. For example, an assay for measuring HbA may comprise calibrant beads comprising known quantities of HbA, thereby enabling signal calibration during an assay (e.g., correlation of signal intensity with the amount of the target biomolecule on a bead).

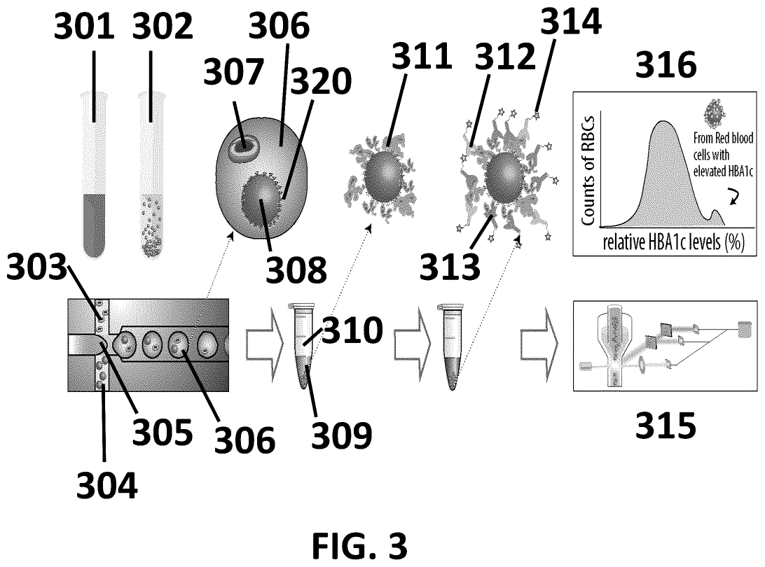

FIG. 3 illustrates a scheme for an assay to identify biomolecule levels from single cells. In this approach, a biological sample comprising cells 301 and sample comprising beads 302 may be input into a droplet generating fluidic device, as described in FIG. 1. The cells and beads may be flown through separate channels (303 and 304, respectively) that meet at a droplet generating junction 305 (e.g., a junction comprising a nozzle that injects oil droplets into a water filled channel), such that the droplets 306 comprise an average of one cell 307 and one bead 308. A plurality or majority of droplets may be empty or may only comprise a bead, and the majority of partitions comprising a cell comprise a single cell and a single bead. The cell may be permeabilized within the droplet.

A bead 308 may comprise a plurality of capture moieties 320. The capture moieties may bind biomolecules from the cell, such as hemoglobin molecules liberated from a permeabilized cell. A plurality of beads 309 may be collected in a partition 310, such as a test tube or well. The partition may collect droplets from the droplet generating fluidic device. A bead may comprise a biomolecule 311 from the cell with which it was partitioned in a droplet. A label 312 and 313 or a plurality of labels may be added to the partition. The labels may selectively label biomolecules from the cell. For example, the plurality of labels may comprise a first antibody that binds HbA and a second antibody that binds HbA1c. A label may comprise a detectable moiety, such as an optically detectable dye molecule. In many cases, a plurality of labels comprises a plurality of unique and distinguishable labels (314). For example, two antibodies with different paratopes may comprise different fluorescent dyes. The beads may be analyzed 315 (e.g., optically analyzed). The analysis may identify or quantify labels bound to biomolecules bound to the beads. A bead may be analyzed individually, thereby providing data 316 corresponding to single cells from the biological sample 301 (e.g., arrow indicates data obtained from red blood cells with elevated HBA1c).

In some cases, the method comprises partitioning the biological sample, wherein at least one first partition comprises one cell from the first plurality of cells and a first bead and at least one second partition comprises one cell from the second plurality of cells and a second bead; measuring a first expression level of a molecule derived from the at least one first partition and a second expression level of a molecule derived from the at least one second partition; and determining a difference between the first expression level and the second expression level. In some cases, the method comprises partitioning the biological sample, wherein at least one first partition comprises one red blood cell from the first plurality of red blood cells and a first bead and at least one second partition comprises one red blood cell from the second plurality of red blood cells and a second bead; measuring a first expression level of a molecule derived from the at least one first partition and a second expression level of a molecule derived from the at least one second partition; and determining a difference between the first expression level and the second expression level.

In some embodiments, the methods of the disclosure comprise detecting at least one labeled biomolecule. In some embodiments, the detecting is by flow cytometry. In some embodiments, the detecting is by fluorosequencing or sequencing by degradation.

### Fluorosequencing

Fluorosequencing (sequencing by degradation) refers to sequencing peptides in a complex protein sample at the level of single molecules. In some embodiments, millions of individual fluorescently labeled peptides are visualized in parallel, monitoring changing patterns of fluorescence intensity as N-terminal amino acids are sequentially removed, and using the resulting fluorescence signatures (fluorosequences) to uniquely identify individual peptides. In some embodiments, amino acids are selectively labeled on immobilized peptides, and the amino acids are subjected to successive cycles of removing the peptide N-terminal residues (Edman degradation) and imaging the corresponding decrease of fluorescent intensity for individual peptide molecules. In some embodiments, amino acids are cleaved using chemical degradation, photochemical degradation, or enzymatic degradation. The methods of the present disclosure are capable of producing patterns sufficiently reflective of the peptide sequences to allow unique identification of a majority of proteins from a species. The resulting stair-step patterns of fluorescence decreases provide positional information of the select amino acid residues. This partial pattern is often sufficient to allow unique identification of the peptide by comparison to a reference proteome. The patterns of cleavage (even for a portion of the protein) provide sufficient information to identify a significant fraction of proteins within a known proteome, i.e. where the sequences of proteins are known in advance. In one embodiment, the single-molecule technologies of the present application allow the identification and absolute quantitation of a given peptide or protein in a biological sample.

In some embodiments, the methods disclosed herein can be used to perform large-scale sequencing (including but not limited to partial sequencing) of single intact peptides (not denatured) at the single molecule level by selective labeling amino acids on immobilized peptides followed by successive cycles of labeling and removal of the peptide amino-terminal amino acids. The methods and systems of the disclosure can identify amino acids in peptides, including peptides comprising unnatural amino acids. In one embodiment, the present disclosure comprises labeling the N-terminal amino acid with a first label and labeling an internal amino acid with a second label. In some embodiments, the labels are fluorescent labels. In other embodiments, the internal amino acid is Lysine. In other embodiments, amino acids in peptides are identified based on the fluorescent signature for each peptide at the single molecule level.

Various aspects of the present disclosure provide compositions and/or methods for peptide fluorosequencing, also called sequencing by degradation. A method consistent with the present disclosure may subject a peptide to fluorosequencing and/or an additional form of analysis. For example, a molecule of hemoglobin may be interrogated for glycation with immunostaining, and/or then subsequently digested and/or subjected to fluorosequencing for sequencing analysis. In one embodiment, the present disclosure provides a massively parallel and rapid method for identifying and quantitating individual peptide and/or protein molecules within a given complex sample.

In some embodiments, the methods of the disclosure comprises: (a) providing a polypeptide, wherein the polypeptide comprises at least one labeled internal amino acid; (b) detecting at least one signal or signal change from the polypeptide to identify at least a portion of a sequence of the polypeptide; and (c) subjecting the polypeptide to conditions sufficient to remove at least one amino acid from the polypeptide. In some embodiments, the at least one amino acid is removed from an N-terminus of the polypeptide. In some embodiments, subsequent to (c), the at least one labeled internal amino acid becomes a labeled terminal amino acid. In some embodiments, the at least one labeled internal amino acid is from a plurality of labeled amino acids, and wherein the at least one signal or signal change comprises a collective signal from the plurality of labeled amino acids. In some embodiments, the plurality of labeled amino acids comprise amino acids with different labels. In some embodiments, the different labels generate signals with different signal patterns.

In some embodiments, the at least one labeled internal amino acid comprises one or more members selected from the group consisting of lysine, glutamate, and aspartate. In some embodiments, the at least one labeled internal amino acid comprises an amino acid having a label covalently attached thereto, which label generates the at least one signal or signal change. In some embodiments, the at least one labeled internal amino acid comprises an amino acid having a dye coupled thereto, which dye generates the at least one signal or signal change. In some embodiments, the at least one signal or signal change is an optical signal. In some embodiments, the at least one signal or signal change is detected with an optical detector having single-molecule sensitivity. In some embodiments, the at least one signal or signal change comprises a plurality of signals of different intensities. In some embodiments, the at least one signal or signal change comprises a plurality of signals of different frequencies or frequency ranges.

In some embodiments, the at least one amino acid is removed from the polypeptide by a degradation reaction. In some embodiments, the degradation reaction is Edman degradation. In some embodiments, the method further comprises processing at least the portion of the sequence against a reference sequence to identify the polypeptide or a protein from which the polypeptide is derived. In some embodiments, the method further comprises, subsequent to (c), (i) identifying the at least the portion of the sequence of the polypeptide to identify the polypeptide, and (ii) using the polypeptide identified in (i) to quantify the polypeptide or a protein from which the polypeptide was derived. In some embodiments, in (a), less than all amino acids of the polypeptide are labeled. In some embodiments, the method further comprises (i) repeating (b) and (c) to detect at least one additional signal or signal change from the polypeptide and (ii) using the at least one signal or signal change and the at least one additional signal or signal change to identify the at least the portion of the sequence.

A characteristic feature of many fluorosequencing methods is coupling amino acid labels to a peptide to be sequenced. A label may be an amino acid specific label (e.g., configured to couple to a specific type of amino acid or a specific set of types of amino acids). A fluorosequencing method may comprise labeling a plurality of types of amino acids with separate, amino acid type specific labels. A fluorosequencing method may comprise labeling one, two, three, four, five, six, or more different types of amino acids residues in a subject peptide or protein. A plurality of amino acid residues may include, for example, an N-terminal amino acid, cysteine, lysine, glutamic acid, aspartic acid, tryptophan, tyrosine, serine, threonine, arginine, histidine, methionine, or any combination thereof. Each of these amino acid residues may be labeled with a different labeling moiety. Multiple amino acid residues may be labeled with the same labeling moiety such as (i) aspartic acid and/or glutamic acid or (ii) serine and/or threonine.

In one embodiment, a method of labeling a peptide comprises: a) providing, i) a peptide having at least one Cysteine amino acid, at least one Lysine amino acid, an N-terminal end, an amino acid having at least one carboxylate side group, a C-terminal end, and at least one Tryptophan amino acid, and ii) a first compound, iii) a second compound, iv) a third compound, v) a fourth compound, and vi) a fifth compound; and b) labeling the Cysteine with the first compound, c) labeling the Lysine with the second compound, d) labeling the N-terminal end with the third compound, e) labeling the carboxylate side group and the C-terminal end with the fourth compound; and f) labeling the Tryptophan with the fifth compound for providing a peptide having specific labels. In one embodiment, b-f are sequential in order from b-f. In one embodiment, the labeling in b-f is performed in one (a single) solution. In one embodiment, b-f are sequential in order from b-f and performed in one solution. In one embodiment, the first compound is iodoacetamide. In one embodiment, the second compound is 2-methylthio-2-imadazoline hydroiodide (MDI). In one embodiment, the third compound is 1-(4,4-dimethyl-2,6-dioxocyclohexylidene)-3-methylbutyl diethyl phosphate (Phos-ivDde). In one embodiment, the fourth compound is selected from the group consisting of benzylamine (BA), 3-dimethylaminopropylamine, and isobutylamine. In one embodiment, the fifth compound is 2,4-dinitrobenzenesulfenyl chloride.

In one embodiment, disclosed herein is a method of treating a peptide comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and the N-terminal amino acid of each peptide labeled with a second label, the second label being different from the first label; b) treating the plurality of immobilized peptides under conditions such that each N-terminal amino acid of each peptide is removed; and c) detecting the first signal for each peptide at the single molecule level. In one embodiment, the second label is attached via an amine-reactive dye. In one embodiment, the second label is selected from the group consisting of fluorescein isothiocyanate, rhodamine isothiocyanate or other synthesized fluorescent isothiocyanate derivative. In one embodiment, portions of the emission spectrum of the first label do not overlap with the emission spectrum of the second label. In one embodiment, the removal of the N-terminal amino acid in b) is done under conditions such that the remaining peptides each have a new N-terminal amino acid. In one embodiment, the method further comprises the d) adding the second label to the new N-terminal amino acids of the remaining peptides. In one embodiment, among the remaining peptides the new end terminal amino acid is Lysine. In one embodiment, the method further comprises the e) detecting the next signal for each peptide at the single molecule level.

In one embodiment, the method further comprises treating the immobilized peptides under conditions such that each N-terminal amino acid of each peptide is removed by an Edman degradation reaction; and detecting the signal for each peptide at the single molecule level. In one embodiment, the label is attached to a fluorophore by a covalent bond. In one embodiment, the fluorophore and the covalent bond is resistant to degradation effects when incubated in an Edman degradation reaction solvent. In some embodiments, the fluorophore is a fluorophore that remains intact and attached to the label during Edman degradation sequencing.

The repetitive detection of signal for each peptide at the single molecule level results in a pattern. The resulting pattern is unique to a single-peptide within the plurality of immobilized peptides. In one embodiment, the single-peptide pattern is compared to the proteome of an organism to identify the peptide, one embodiment, the intensity of the labels are measured amongst the plurality of immobilized peptides. In some embodiments, the peptides are immobilized via Cysteine residues. In some embodiments, the detecting in c) is done with optics capable of single-molecule resolution. In a specific embodiment, one or more of the plurality of peptides comprises one or more unnatural amino acids.

In some embodiments, the emission spectrum of the first label do not overlap with the emission spectrum of the second label. In some embodiments, the removal of the N-terminal amino acid in b) is done under conditions such that the remaining peptides each have a new N-terminal amino acid. In one embodiment, the method further comprises d) adding the second label to the new N-terminal amino acids of the remaining peptides. In some embodiments, among the remaining peptides, the new end terminal amino acid is Lysine. In one embodiment, the method further comprises e) detecting the next signal for each peptide at the single molecule level. In one embodiment, the intensity of the first and second labels are measured amongst the plurality of immobilized peptides. In some embodiments, the peptides are immobilized via Cysteine residues. In some embodiments, the detecting in c) is done with optics capable of single-molecule resolution. In one embodiment, one or more of the plurality of peptides comprises one or more unnatural amino acids. In one embodiment, the unnatural amino acids comprises moieties selected from the group consisting of hydroxycarboxylates, aldehydes, thiols, and olefins. In one embodiment, one or more of the plurality of peptides comprises one or more beta amino acids.

In one embodiment, the method further comprises treating an immobilized peptide (e.g., a support or bead) under conditions such that each N-terminal amino acid of each peptide is removed by an Edman degradation reaction; and detecting the signal for each peptide at the single molecule level. In some embodiments, removing the N-terminal amino acid and the detecting are successively repeated from about 1 time to about 5 times, from about 5 times to about 10 times, from about 10 times to about 20 times, from about 20 times to about 30 times, from about 30 times to about 40 times, from about 40 times to about 50 times, from about 50 times to about 60 times, from about 60 times to about 70 times, from about 70 times to about 80 times, from about 80 times to about 90 times, or from about 90 times to about 100 times. In some embodiments, removing the N-terminal amino acid and the detecting are successively repeated at least about 5 times, at least about 10 times, at least about 20 times, at least about 30 times, at least about 40 times, at least about 50 times, at least about 60 times, at least about 70 times, at least about 80 times, at least about 90 times, or at least about 100 times. In some embodiments, removing the N-terminal amino acid and the detecting are successively repeated about 5 times, about 10 times, about 20 times, about 30 times, about 40 times, about 50 times, about 60 times, about 70 times, about 80 times, about 90 times, or about 100 times. In some embodiments, removing the N-terminal amino acid and the detecting are successively repeated at most about 5 times, at most about 10 times, at most about 20 times, at most about 30 times, at most about 40 times, at most about 50 times, at most about 60 times, at most about 70 times, at most about 80 times, at most about 90 times, or at most about 100 times.

A label may comprise a detectable moiety. The detectable moiety (i.e., label) may be optically detectable (e.g., fluorescent, phosphorescent, luminescent, or light absorbing). The detectable moiety may be electrochemically detectable (e.g., a redox active moiety with a characteristic oxidation or reduction potential). The detectable moiety may comprise a mass tag (e.g., for identification with mass spectrometry. A detectable moiety may identify a label to which it is attached. A plurality of labels may comprise a plurality of detectable moieties which identify labels of the plurality of labels by their type. For example, a method may comprise a plurality of types of labels configured to couple to different amino acids, each comprising a different detectable moiety that uniquely identifies the label by its type.

Labeling specificity can be a major challenge for a fluorosequencing method. In many cases, a label may comprise reactivity toward a plurality of amino acid types. For example, some maleimide labels can react with cysteine, lysine, and/or N-terminal amines. Discriminating between similarly reactive amino acid residues can require precise ordering of labeling steps. In the above maleimide example, lysine may be discriminated from cysteine by first reacting cysteine with a cysteine specific labeling step (e.g., iodoacetamide coupling at pH 7-8), thereby preventing further cysteine labeling in a subsequent lysine labeling step. A method may comprise cysteine labeling prior to lysine labeling. A method may comprise cysteine labeling prior to aspartate and/or glutamate labeling. A method may comprise cysteine labeling prior to tryptophan labeling. A method may comprise cysteine labeling prior to tyrosine labeling. A method may comprise cysteine labeling prior to serine and/or threonine labeling. A method may comprise cysteine labeling prior to histidine labeling. A method may comprise cysteine labeling prior to arginine labeling. A method may comprise lysine labeling prior to glutamate labeling. A method may comprise lysine labeling prior to aspartate labeling. A method may comprise lysine labeling prior to tryptophan labeling. A method may comprise lysine labeling prior to tyrosine labeling. A method may comprise tyrosine labeling prior to lysine labeling. A method may comprise lysine labeling prior to serine and/or threonine labeling. A method may comprise lysine labeling prior to arginine labeling. A method may comprise carboxylate side chain (e.g., glutamate and/or aspartate side chain) labeling prior to tryptophan labeling. A method may comprise carboxylate side chain (e.g., glutamate and/or aspartate side chain) labeling prior to tyrosine labeling. A method may comprise carboxylate side chain (e.g., glutamate and/or aspartate side chain) labeling prior to serine labeling. A method may comprise carboxylate side chain (e.g., glutamate and/or aspartate side chain) labeling prior to serine and/or threonine labeling. A method may comprise carboxylate side chain (e.g., glutamate and/or aspartate side chain) labeling prior to histidine labeling. A method may comprise carboxylate side chain (e.g., glutamate and/or aspartate side chain) labeling prior to arginine labeling. A method may comprise C-terminal carboxylate labeling prior to lysine labeling. A method may comprise C-terminal carboxylate labeling prior to tyrosine labeling. A method may comprise C-terminal carboxylate labeling prior to histidine labeling. A method may comprise C-terminal carboxylate labeling prior to tryptophan labeling. A method may comprise C-terminal carboxylate labeling prior to glutamate and/or aspartate labeling. A method may comprise C-terminal carboxylate labeling prior to serine and/or threonine labeling. A method may comprise at least 2, at least 3, at least 4, at least 5, or at least 6 amino acid labeling steps performed in a sequence configured to minimize or prevent label cross-reactivity (e.g., labeling more than the intended type or types of amino acids). A method may comprise 2, 3, 4, 5, or 6 amino acid labeling steps performed in a sequence configured to minimize or prevent label cross-reactivity (e.g., labeling more than the intended type or types of amino acids).

Fluorosequencing may comprise removing peptides through techniques such as Edman degradation following or preceding subject peptide detection. Sequential peptide removal may generate sequence or position-specific information. For example, a reduction in fluorescence following an N-terminal amino acid removal step may indicate that a labeled amino acid, and/or thus that a specific type of amino acid, was disposed at a peptide N-terminal. Removal of each amino acid residue can be carried out with a variety of different techniques including Edman degradation and/or proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide chain. In situations where Edman degradation is used, the amino acid residue at the N-terminus of the peptide chain is removed.

In one embodiment, the label is attached to a fluorophore by a covalent bond. In one embodiment, the fluorophore and the covalent bond is resistant to degradation effects when incubated in an Edman degradation reaction solvent. A labeling moiety used in the instant application may be configured to withstand conditions for removing one or more of the amino acid residues. Some non-limiting examples of potential labeling moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and/or (5)6-napthofluorescein. In some embodiments, a labeling moiety is tetramethylrhodamine, Si-Rhodamine, Rhodamine B, Rhodamine B N, N′-dimethylethylenediamine, Rhodamine B sulfenyl chloride, Alexafluor555, Alexa Fluor 405, Atto647N, (5)6-napthofluorescein, variants and derivations thereof, etc. In one embodiment, the fluorophore is selected from the group consisting of tetramethylrhodamine, Si-Rhodamine, Rhodamine B, Rhodamine B N, N′-dimethyl ethylenediamine, Rhodamine B sulfenyl chloride, Alexafluor555, Alexa Fluor 405, Atto647N, (5)6-napthofluorescein, variants and derivations thereof. The labeling moiety may be a fluorescent peptide or protein or a quantum dot. In some embodiments, two-color single molecule peptide sequencing reactions can be used to identify and quantify biomolecules by using two or more fluorescent molecules.

In some embodiments, amino acids can be removed from the carboxy terminus of a biomolecule, revealing C-terminal sequences instead of N-terminal sequences. In some embodiments, an engineered carboxypeptidase is used to mimic Edman degradation.

In some embodiments, the methods disclosed herein comprise identifying amino acids in peptides, comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and the N-terminal amino acid of each peptide labeled with a second label, the second label being different from the first label and selected from the group consisting of Alexa fluor dyes and Atto dyes, wherein a subset of the plurality of peptides comprise an N-terminal Lysine having both the first and second label; b) treating the plurality of immobilized peptides under conditions such that each N-terminal amino acid of each peptide is removed by an Edman degradation reaction; and c) detecting the first signal for each peptide at the single molecule level under conditions such that the subset of peptides comprising an N-terminal Lysine is identified. It is preferred that the removal of the N-terminal amino acid in b) is done under conditions such that the remaining peptides each have a new N-terminal amino acid. The present disclosure further contemplates in one embodiment, a method of identifying amino acids in peptides, comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and the N-terminal amino acid of each peptide labeled with a second label, the second label being different from the first label and selected from the group consisting of Alexa fluor dyes and Atto dyes, wherein a subset of the plurality of peptides comprise an N-terminal acid that is not Lysine; b) treating the plurality of immobilized peptides under conditions such that each N-terminal amino acid of each peptide is removed by an Edman degradation reaction; and c) detecting the first signal for each peptide at the single molecule level under conditions such that the subset of peptides comprising an N-terminal amino acid that is not Lysine is identified. It is preferred that the removal of the N-terminal amino acid in b) is done under conditions such that the remaining peptides each have a new N-terminal amino acid. It is preferred that the peptides are immobilized via Cysteine residues. In one embodiment, one or more of the plurality of peptides comprises one or more unnatural amino acids. In one embodiment, the unnatural amino acids comprise moieties selected from the group consisting of hydroxycarboxylates, aldehydes, thiols, and olefins, one embodiment, one or more of the plurality of peptides comprises one or more beta amino acids.

Detecting the immobilized peptide may comprise capturing an image comprising the peptide. The image may comprise a spatial address specific to the peptide. A plurality of peptides may be detected in a single image, wherein one or more of the peptides may comprise a spatial address within the image. The surface may be optically transparent across the visible spectrum and/or the infrared spectrum. The surface may possess a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 μm thick, between 1 and 5 μm thick, between 2 and 10 μm thick, between 5 and 20 μm thick, between 20 and 50 μm thick, between 50 and 200 μm thick, between 200 and 500 μm thick, or greater than 500 μm in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and/or plasma enhanced chemical vapor deposition) and/or functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and/or modified targets for selection. Alternatively, an aminosilane modified surfaces may be used in the methods described herein. The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein may involve imaging the peptide or protein to determine the presence of one or more labeling moieties (e.g., amino acid labels) coupled to the peptide. The sequencing technique may comprise imaging a plurality of peptides or proteins to determine the presence of one or more labeling moieties on individual peptides from among the plurality of peptides. The sequencing technique may comprise imaging from about 10³ to about 10⁴, from about 10⁴ to about 10⁵, from about 10⁵ to about 10⁶, from about 10⁶ to about 10⁷, or from about 10⁷ to about 10⁸ proteins or peptides. The sequencing technique may comprise imaging at least about 10³, at least about 10⁴, at least about 10⁵, at least about 10⁶, at least about 107, or at least about 10⁸ or more proteins or peptides (e.g., imaging a portion of a surface comprising at least about 10³ to at least about 10⁸ proteins or peptides). The sequencing technique may comprise imaging about 10³, about 10⁴, about 10⁵, about 10⁶, about 10⁷, or about 10⁸ or more proteins or peptides (e.g., imaging a portion of a surface comprising about 10³ to about 10⁸ proteins or peptides). The sequencing technique may comprise imaging at most about 10³, at most about 10⁴, at most about 10⁵, at most about 10⁶, at most about 10⁷, or at most about 10⁸ or more proteins or peptides (e.g., imaging a portion of a surface comprising at most about 10³ to at most about 10⁸ proteins or peptides).

These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (wherein ‘K’ denotes lysine, ‘D’ denotes aspartate, ‘Y’ denotes tyrosine, ‘A’ denotes alanine, and ‘G’ denotes glycine), and/or may comprise labels coupled to each lysine and/or tyrosine residue. A first image comprising the C-terminal immobilized peptide may indicate the presence of two lysines and/or one tyrosine in the peptide. The N-terminal amino acid may be removed (e.g., by Edman degradation), such that a second image comprising the C-terminal immobilized peptide may indicate the presence of one lysine and/or one tyrosine in the peptide. This process may be repeated until a sequence of KXXYXXXXXXXKX is identified for the peptide, wherein ‘X’ indicates a non-lysine, non-tyrosine amino acid, ‘K’ indicates a lysine, and ‘Y’ indicates a tyrosine. A method of the present disclosure can identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and/or comparing these locations to known peptide sequences, which may identify the entire list of amino acid residues in the peptide sequence. For example, identifying the positions of the lysines and/or cysteines in a 40 amino acid fragment of a human protein may uniquely identify the protein (e.g., only one human protein contains the specific pattern of lysine and/or cysteine residues identified in the 40 amino acid fragment).

An imaging method may involve a variety of different spectrophotometric and/or microscopy methods, such as fluorimetry, diffuse reflectance, interferometric scattering, Raman, resonance enhanced Raman, infrared absorbance, visible light absorbance, ultraviolet absorbance, and/or fluorescence. In some embodiments, a microscope equipped with total internal reflection illumination and/or an intensified charge-couple device (CCD) detector may be used for imaging. Depending on the absorption and emission spectra of fluorescent Edman labels employed, appropriate filters can be used to record the emission intensity of the labels. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide. Such imaging methods may be used to determine the presence or absence of a label on a specific peptide sequence. After repeated cycles of removing an amino acid residue and/or imaging a subject peptide, the position of the labeled amino acid residue can be determined in the peptide.

For each Edman cycle, the fluorescence intensity of a label is recorded after each cleavage step. The loss and uptake of a label after each cleavage step and coupling step serves as a 1) counter for the number of amino acid residues removed, and 2) an internal error control indicating the successful completion of each round of Edman degradation for each immobilized peptide.

Following image processing to filter noise and identify the location of peptides, and to map the locations of the same peptides across the set of collected images, intensity profiles for labels are associated with each peptide as a function of Edman cycle. The label intensity profile of each error free peptide sequencing reaction is transformed into a binary sequence in which a “1” precedes a drop in fluorescence intensity and its location (i.e., position within the binary sequence). Identifies the number of Edman cycles performed. A database of predicted potential proteins is used as a reference database. The binary intensity profile of each peptide, as generated from the single molecule microscopy, is then compared to the entries in the simulated peptide database. Quantification can be accomplished by counting peptides derived from each protein observed.

### Applications

Aspects of the present disclosure provide a method for distinguishing a first plurality of cells from a second plurality of cells in a biological sample. In many cases, the method comprises quantifying the total or relative abundances of the first and second cells in the biological sample. In particular cases, the first plurality and second plurality of cells may comprise different characteristics or properties, and the method may comprise measuring the differences in characteristics or properties from a sample to distinguish the first plurality of cells from the second plurality of cells. In some cases, three or more pluralities of cells may be distinguished.

In some embodiments, the cell is a red blood cell, a stem cell, a bone cell, a muscle cell, fat cell, skin cell, nerve cell, endothelial cell, sex cell, pancreatic cell, or cancer cell. In some embodiments, the cell is a red blood cell. In some embodiments, the cell is a skin cell. In some embodiments, the cell is a muscle cell. In some embodiments, the cell is a endothelial cell. In some embodiments, the cell is a cancer cell.

Aspects of the present disclosure provide a method for distinguishing a first plurality of red blood cells from a second plurality of red blood cells in a biological sample. In many cases, the method comprises quantifying the total or relative abundances of the first and second red blood cells in the biological sample. In particular cases, the first plurality and second plurality of red blood cells may comprise different hemoglobin profiles (e.g., different ratios of hemoglobin types, such as HbA and/or HbA1c), and the method may comprise measuring the hemoglobin profiles of red blood cells from a sample to distinguish the first plurality of red blood cells from the second plurality of red blood cells. In some cases, three or more pluralities of red blood cells may be distinguished.

A particular application of relevance for such techniques is identifying autologous blood transfusion in professional sports. As the past couple of decades have provided a range of detection techniques for steroid and/or growth hormone use, athletes have progressively turned to “blood doping”, the practice of transfusing blood prior to increase red blood cell count and thus raise oxygen carrying capacity. Blood doping has remained an elusive practice, as average blood biomarker levels often shift negligibly as a result of transfusion. Therefore, methods for identifying biomarkers on the single cell level may be particularly well suited for identifying blood doping in athletes.

Various aspects of the present disclosure provide methods for distinguishing a first plurality of cells from a second plurality of cells in a biological sample. In many cases, the methods quantitate the first plurality and the second plurality of cells. For example, a method may determine a ratio between the first plurality and second plurality of cells in a biological sample. In some cases, the methods comprise partitioning the biological sample, resulting in at least one first partition comprising one cell from the first plurality of cells and one bead, and at least one second partition comprising one cell from the second plurality of cells and one bead. In some cases, the methods comprise measuring a first expression level of a molecule from the at least one first partition and measuring a second expression level of a molecule from the at least one second partition and determining a difference between the first expression level and the second expression level. In some cases, the first expression level and second expression level correspond to the same type of molecule or different types of molecules.

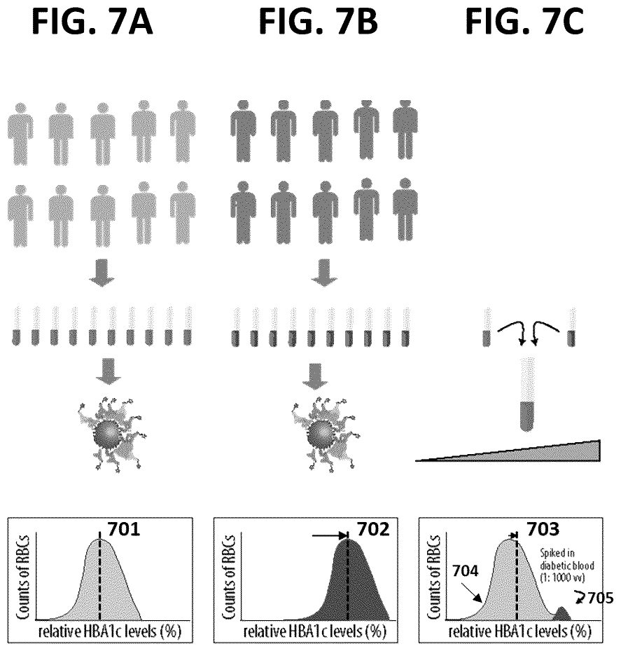

The first and second expression levels may be indicative of a biological state or condition. For example, glycated hemoglobin levels may be elevated in autologously transfused red blood cells but may also be indicative of a number of metabolic disorders, such as diabetes. As is illustrated in FIG. 7, a challenge in identifying autologous blood transfusion is that bulk hemoglobin analysis (e.g., averaged from a collection of cells) may be unable to differentiate between a subpopulation of red blood cells with spiked glycated hemoglobin levels and/or a small increase in glycated hemoglobin levels across all cells in a sample. FIG. 7A-7B depict two separate human populations (top) with different red blood cell HbA1c:HbA ratios (bottom graphs). The bottom graphs provide histograms of relative HbA1c to HbA levels in blood, with the x-axes providing the HbA1c to HbA ratios and/or the y-axes providing cell counts at each ratio. As may be seen from these graphs. the two human populations may be readily distinguished by their average red blood cell HbA1c levels, which are depicted by the dotted lines 701 and 702. However, upon addition of a small amount of blood from the second population to a sample of blood from the first population (as is depicted in FIG. 7C), the average HbA1c:HbA ratio of blood cells in the sample (dashed line 703) may only shift a small amount, such that the measured average does not distinguish the spiked sample from a pure sample from the first population. However, by measuring HbA1c:HbA ratios on a single cell level, two separate populations 704 and 705 may readily be distinguished. Thus, the methods of the present disclosure provide measuring expression levels on a single cell basis. In many cases, the presence of cellular subpopulations may be resolved, allowing the biological state of the patient to be more clearly defined than if average biomolecule levels were measured for pluralities of cells.

Red blood cells contain a large number of expression products that may serve as markers for a wide range of biological states. In some cases, a measured expression level may refer to the amount of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), oxidized proteins, or glycosylated proteins. In some cases, the molecule is HbA. In some cases, the molecule is HbA1c. In the case of autologous blood transfer, HbA1c levels may be raised relative to HbA levels. However, the abundance of autologously transfused red blood cells is often low compared to native red blood cell levels. Therefore, autologous blood transfer often provides a small shift in average red blood cell HbA1c levels that is insufficient for determining whether a sample comprises autologously transferred blood. However, when HbA1c levels are measured at a single cell level, the population of red blood cells from the autologously transferred blood may easily be distinguished from the population of native blood cells, and thus may serve as an accurate diagnostic marker.

In some cases, the methods comprise lysing the one red blood cell from the first plurality of red blood cells and the one red blood cell from the second plurality of red blood cells subsequent to partitioning the biological sample. In some cases, the methods comprise fragmenting HbA from the first partition and the second partition prior to measuring expression levels. In some cases, the methods comprise immobilizing HbA from the first partition and second partition to the first bead and the second bead respectively prior to measuring expression levels.

The first and second expression levels may be measured with a variety of methods. The measurement may be specific for a class of molecules (e.g., any form of hemoglobin), or for a specific form of a biomolecule. For example, a measurement may specifically detect a protein with a particular post-translational modification, such as N-terminal valine glycated HbA. In some cases, the measuring comprises detecting a post-translational modification that comprises oxidation, glycation, or glycosylation.

In some cases, the measuring comprises coupling a first antibody to an expression product. Further, the measuring may comprise coupling a second antibody to the first antibody expression product. In some cases, the first antibody is specific for a type of protein and/or the second antibody is specific for a particular post-translational modification. In this way, both the expression level of a type of protein and/or the prevalence of the post-translational modification may be measured. For example, the measuring may comprise coupling a first antibody to a first epitope on HbA and/or a second antibody to a particular post-translational modification on HbA, such as N-terminal valine glycation.

### Kits

Various aspects of the present disclosure provide kits for assaying a molecule from a cell. The kit may comprise reagents for partitioning a cell. The kit may comprise reagents for detecting a biomolecule from the cell. The kit may comprise reagents or components compatible with a commercial sell sorting instrument. In some cases, a kit comprises a plurality of beads and/or a plurality of detection moieties for measuring an expression level of a molecule from a cell. In some cases, the cell is a red blood cell. In some cases, a bead comprises a protein capture moiety. In some cases, the protein capture moiety comprises maleimide. In some cases, a bead is magnetic (e.g., is a polymer coated SPION). In some cases, the kit comprises instructions for using the plurality of beads and/or the plurality of detection moieties to assay the molecule from the cell.

In some embodiments, disclosed herein is a kit for assaying a biomolecule from a cell, comprising: (a) a plurality of beads, wherein at the plurality of beads comprise a protein capture moiety; (b) a plurality of detection moieties for measuring an expression level of the biomolecule from the cell; and/or (c) instructions for using the plurality of beads and the plurality of detection moieties to assay the biomolecule from the cell. In some embodiments, the plurality of beads comprise a protein capture moiety comprising a reactive group that forms a covalent bond with the target biomolecule. In some embodiments, the plurality of beads comprise a protein capture moiety comprising a maleimide group. In some embodiments, the plurality of beads comprise a protein capture moiety comprising a haloacetyl group. In some embodiments, the plurality of beads comprise a protein capture moiety comprising a pyridyl disulfide group.

In some embodiments, disclosed herein is a kit for quantifying HbA and/or HbA1c levels in a single red blood cell, comprising: (i) a plurality of beads, wherein at least one bead comprises a maleimide capture moiety; (ii) an HbA1c targeting antibody comprising a first label; (iii) an HbA targeting antibody comprising a second label; (iv) tris(2-carboxyethyl)phosphine (TCEP); (v) lysis buffer; (vi) a denaturing reagent; (vii) bovine serum albumin; and/or (viii) instructions for using the plurality of beads with the antibodies and/or reagents to quantify HbA and/or HbA1c levels in a single red blood cell.

The kit may comprise additional components. In some cases, the kit comprises a lysis reagent. In some cases, the kit comprises a denaturing reagent. In some cases, the denaturing reagent comprises a detergent. In some cases, the kit comprises a protease. In some cases, the kit comprises a calibrant. In some cases, the calibrant comprises a bead functionalized with a determined amount of a target biomolecule. For example, an assay for measuring HbA1c may comprise calibrant beads functionalized with known quantities of HbA1c, thereby allowing the signals (e.g., the signal intensities) from an assay to be calibrated to the quantities of the target biomolecule obtained. In some cases, the kit comprises a reductant. In some cases, the kit comprises a coupling reagent. In some cases, the coupling agent is an enzymatic coupling agent. In some cases, the kit comprises a buffer. In some cases, the buffer is a lysis buffer. In some cases, a reagent (e.g., a denaturing reagent) is provided in a buffer. In some cases, two reagents are provided in different buffers. In some cases, the molecule is lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin A1c (HbA1c), or hemoglobin F (HbF) In some cases, the molecule is hemoglobin. In some cases, the molecule is HbA or HbA1c.

In some cases, the cell is a red blood cell. In some cases, the biomolecule is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), and hemoglobin F (HbF). In some cases, the plurality of beads are magnetically labelled. In some cases, the plurality of beads comprise a mean diameter of from about 15 nm to about 5 μm. In some cases, the plurality of beads comprise a mean diameter of from about 1 μm to about 5 μm. In some cases, the plurality of beads comprises at least 10⁶ capture moieties. In some cases, the plurality of beads comprises at least 10⁷ capture moieties.

In some cases, the plurality of beads comprise a barcode. In some cases, the barcode is a deoxyribonucleic acid (DNA), a ribonucleic acid (RNA), a peptide nucleic acid (PNA), a polypeptide, or a combination thereof. In some cases, the barcode is an oligopeptide comprising from about 2 amino acids to about 30 amino acids. In some cases, the barcode comprises glutamic acid, aspartic acid, cysteine, methionine, lysine, arginine, tyrosine, tryptophan, and/or phenylalanine. In some cases, the barcode comprises a non-natural amino acid. In some cases, the plurality of detection moieties comprises an amino acid specific label.

In some cases, the plurality of detection moieties comprises an amino acid specific label. The amino acid specific label may comprise a moiety that binds to a specific type (e.g., histidine) or class (carboxylic acid side chain bearing) amino acids. The amino acid specific label may comprise a moiety that binds to a specific post translational modification, such as a phosphoryl group or a particular glycosylation pattern. The moiety of the amino acid specific label may covalently attach to a target amino acid, and/or may comprise a small molecule, such as a maleimide. The amino acid specific label may be optically detectable. In some cases, the amino acid specific label comprises an optically active label, such as a dye, for detection. In some cases, the amino acid specific label is optically detectable. In some cases, the plurality of detection moieties comprises a first antibody and a second antibody.

In some cases, the plurality of detection moieties comprises an antibody. In some cases, the plurality of detection moieties comprises a first antibody and a second antibody. In some cases, the first antibody and the second antibody comprise paratopes for different forms of hemoglobin. For example, the first antibody may comprise a paratope for HbA and the second antibody may comprise a paratope for glycated HbA (HbA1c).

In some cases, a plurality of amino acid specific labels is provided with different optically detectable labels. For example, a set of 5 amino acid specific labels may comprise 5 separate dyes with different fluorescence profiles, thereby allowing the 5 labels to be distinguished by fluorescence. In some cases, a kit comprises a first antibody with a first label and a second antibody with a second label. In some cases, the first label and second labels comprise different optically detectable moieties.

In some cases, the kit enables quantification of HbA and/or HbA1c levels in a single red blood cell. In some cases, the kit comprises a plurality of beads, an HbA1c targeting antibody comprising a first label, an HbA targeting antibody comprising a second label, a reducing agent, lysis buffer, and/or a denaturing reagent. In some cases, at least one bead comprises a maleimide capture moiety. In some cases, the kit comprises bovine serum albumin. In some cases, the reducing reagent comprises tris(2-carboxyethyl)phosphine (TCEP). In some cases, the kit comprises instructions for using the plurality of beads with the antibodies and/or reagents to quantify HbA and/or HbA1c levels in a single red blood cell.

### Computer Systems

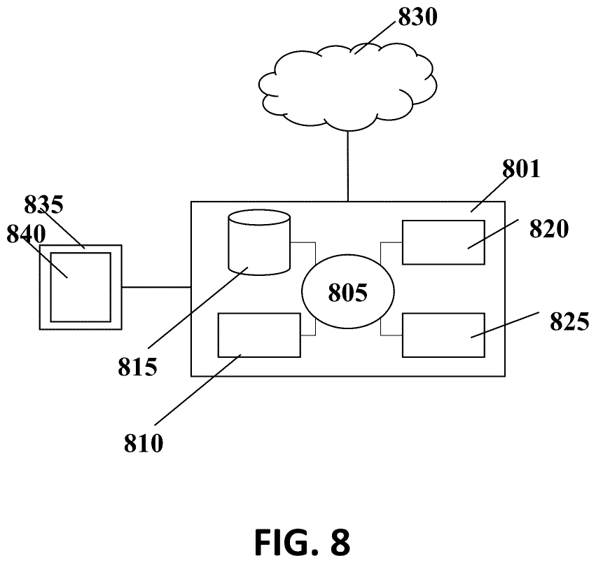

The present disclosure provides computer systems that are programmed to implement methods of the disclosure. FIG. 8 shows a computer system 801 that is programmed or otherwise configured to implement methods or parts of methods disclosed herein, including compiling, analyzing, and/or displaying data obtained through the present methods. The computer system 801 may regulate various aspects of the present disclosure, such as, for example, controlling cell partitioning and/or optical imaging devices. The computer system 801 may be an electronic device of a user or a computer system that is remotely located with respect to the electronic device. The electronic device may be a mobile electronic device.

The computer system 801 includes a central processing unit (CPU, also “processor” and/or “computer processor” herein) 805, which may be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and/or peripheral devices 825, such as cache, other memory, data storage and/or electronic display adapters. The memory 810, storage unit 815, interface 820 and/or peripheral devices 825 are in communication with the CPU 805 through a communication bus (solid lines), such as a motherboard. The storage unit 815 may be a data storage unit (or data repository) for storing data. The computer system 801 may be operatively coupled to a computer network (“network”) 830 with the aid of the communication interface 820. The network 830 may be the Internet, an internet and/or extranet, or an intranet and/or extranet that is in communication with the Internet. The network 830 in some cases is a telecommunication and/or data network. The network 830 may include one or more computer servers, which may enable distributed computing, such as cloud computing. The network 830, in some cases with the aid of the computer system 801, may implement a peer-to-peer network, which may enable devices coupled to the computer system 801 to behave as a client or a server.

The CPU 805 may execute a sequence of machine-readable instructions, which may be embodied in a program or software. The instructions may be stored in a memory location, such as the memory 810. The instructions may be directed to the CPU 805, which may subsequently program or otherwise configure the CPU 805 to implement methods of the present disclosure. Examples of operations performed by the CPU 805 may include fetch, decode, execute, and/or writeback.

The CPU 805 may be part of a circuit, such as an integrated circuit. One or more other components of the system 801 may be included in the circuit. In some cases, the circuit is an application specific integrated circuit (ASIC).

The storage unit 815 may store files, such as drivers, libraries and/or saved programs. The storage unit 815 may store user data, e.g., user preferences and/or user programs. The computer system 801 in some cases may include one or more additional data storage units that are external to the computer system 801, such as located on a remote server that is in communication with the computer system 801 through an intranet or the Internet.

The computer system 801 may communicate with one or more remote computer systems through the network 830. For instance, the computer system 801 may communicate with a remote computer system of a user (e.g., a fluorimeter or a cell sorting device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 801 via the network 830.

Methods as described herein may be implemented by way of machine (e.g., computer processor) executable code stored on an electronic storage location of the computer system 801, such as, for example, on the memory 810 or electronic storage unit 815. The machine executable or machine readable code may be provided in the form of software. During use, the code may be executed by the processor 805. In some cases, the code may be retrieved from the storage unit 815 and stored on the memory 810 for ready access by the processor 805. In some situations, the electronic storage unit 815 may be precluded, and/or machine-executable instructions are stored on memory 810.

The code may be pre-compiled and/or configured for use with a machine having a processer adapted to execute the code or may be compiled during runtime. The code may be supplied in a programming language that may be selected to enable the code to execute in a pre-compiled or as-compiled fashion.

Aspects of the systems and methods provided herein, such as the computer system 801, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and/or the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and/or electromagnetic waves, such as used across physical interfaces between local devices, through wired and/or optical landline networks and/or over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and/or fiber optics, including the wires that comprise a bus within a computer system. Carrier-wave transmission media may take the form of electric or electromagnetic signals, or acoustic or light waves such as those generated during radio frequency (RF) and/or infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and/or EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 may include or be in communication with an electronic display 835 that comprises a user interface (UI) 840 for providing, for example, orders and/or options for controlling flow rates in a cell sorting device. Examples of UI's include, without limitation, a graphical user interface (GUI) and/or web-based user interface.

Methods and systems of the present disclosure may be implemented by way of one or more algorithms. An algorithm may be implemented by way of software upon execution by the central processing unit 805. The algorithm may, for example, determine a correlation using 1 quadratic discriminant analysis (QDA), Support Vector Machine (SVM), linear discriminant analysis (LDA), quadratic discriminant analysis (QDA), Naive Bayes, Random Forest, or any other suitable method.

### EXAMPLES

### Example 1: Functionalized Bead for Protein Capture

This example provides a bead comprising a plurality of reactive sites. The reactive sites provide a modularity to the beads, by allowing them to bind capture moieties designed to bind a biomolecule of interest. In this way, a bead comprising capture moieties may be provided as a multifunctional reagent (e.g., in a kit), and may subsequently be functionalized for a specific assay.

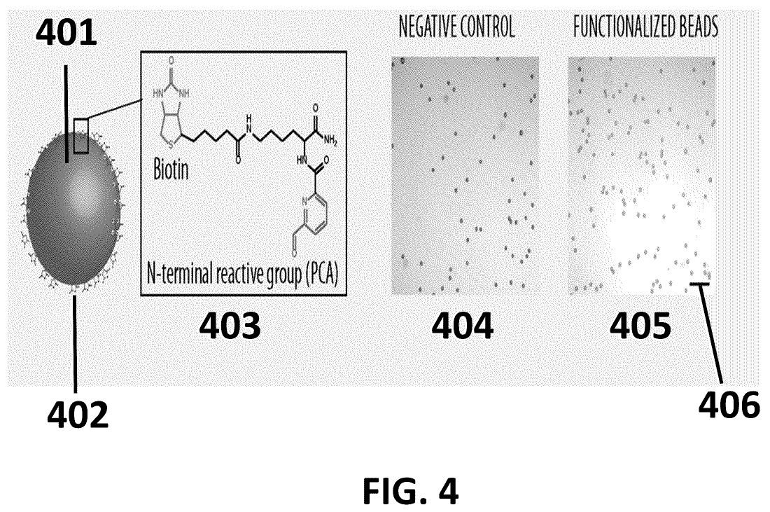

FIG. 4 illustrates a reactive site and capture moiety (402) functionalized bead (401), along with fluorescence microscope images depicting green fluorescent protein (GFP) capture on non-capture moiety functionalized beads and capture moiety functionalized beads. 20 μm beads 401 comprise a plurality of streptavidin reactive sites 402. A first set of beads were functionalized with capture moieties 403 comprising biotin (for binding to the streptavidin on the beads) and the protein capture reagent PCA, which were capable of binding protein N-termini. A second set of beads were not coupled to capture moieties. The first and second sets of beads were then incubated with 1 μmg/mL GFP, washed, and imaged under a microscope. Image 404 shows results with unfunctionalized (lacking capture moieties) beads, provided as overlaid bright-field and FITC channel images. Image 405 shows results with capture moiety functionalized beads, provided as overlaid bright-field and FITC channel images. The scale bar 406 depicts 200 μm. The image of the capture moiety functionalized bead provides considerably higher fluorescence intensity than the unfunctionalized beads, demonstrating that capture moiety, and in particular PCA functionalized beads are capable of binding proteins.

### Example 2: Lab Integrated Single Cell Assay

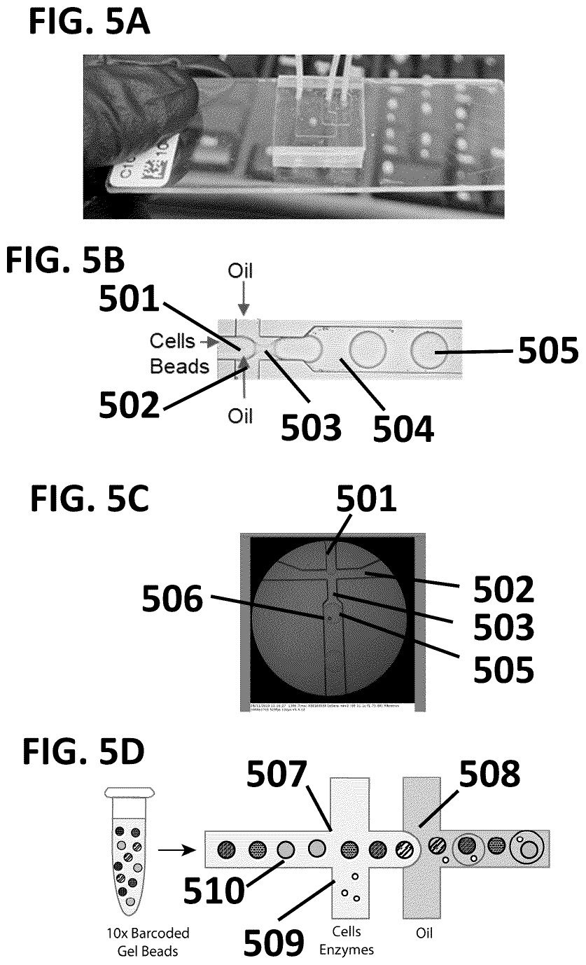

FIG. 5A-5D show an assay consistent with the present disclosure that may be performed on two instruments, along with a design for a microfluidic chip used in the disclosure.

FIG. 5A provides a view of a microfluidic chip capable of forming water-in-oil droplets comprising cells and beads. FIG. 5B provides a microscope image of a microfluidic junction in the microfluidic chip of FIG. 5A, in which the water-in-oil droplets are formed. The microfluidic junction 501 comprises a channel 502 through which aqueous media comprising cells and beads flows in, two channels 502 through which oil flows in, and a narrow opening 503 leading to an exit channel 504. The narrow opening provides uniformity to the droplets 505 formed at the junction. FIG. 5C provides a microscope image of the microfluidic junction, in which a bead 506 may be seen in a droplet 505.

FIG. 5D provides a schematic of the droplet formation at the microfluidic junction. A second microfluidic junction 507 is disposed upstream to the microfluidic junction 508 in which water-in-oil droplets form. At this junction, cells and reagents are flown in through a first channel 509 to converge with beads flown in through a second channel 510. The flow rates and concentrations in each channel are optimized so that an average droplet comprises one bead and one cell.

### Example 3: HbA and HbA1c Calibrant Bead Set

This example provides a set of beads that may be used as calibrants in an assay for HbA1c. This calibrant set comprises four solutions with 5.0%, 7.9%, 10.9% and 13.7% HbA1c (relative to total hemoglobin) provided in dried, lyophilized form, and which may be reconstituted with buffer (e.g., lysis buffer). Calibrant beads are then generated from the reconstituted HbA1c samples through incubation different quantities (10⁶, 10⁷, or 10⁸) of maleimide functionalized beads.

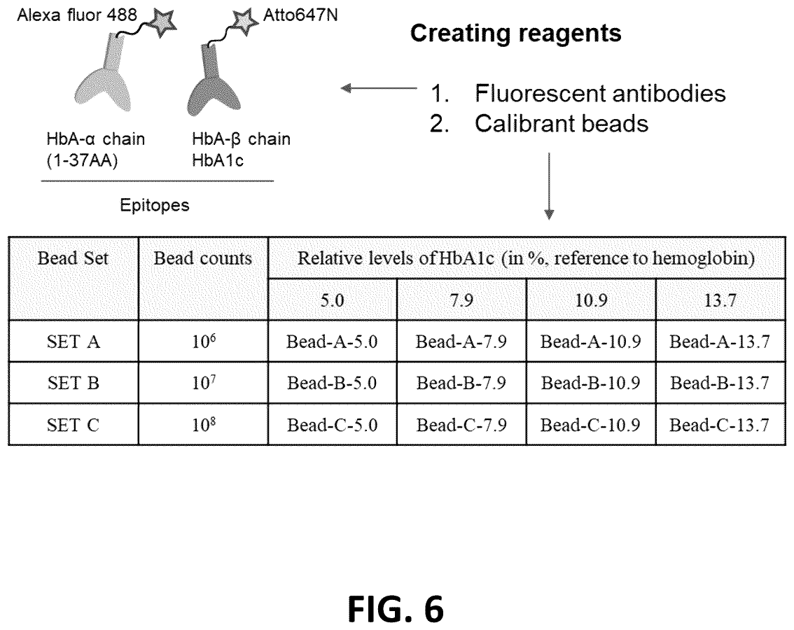

FIG. 6 summarizes the bead counts and HbA1c levels used to generate each calibrant. Two fluorescent antibody epitopes are generated: 1) HbA-α chain (1-37AA) with Alexa fluor 488; and 2) HbA-β chain HbA1c with Atto647N. Three sets of beads are generated with relative levels of HbA1c (in % reference to hemoglobin) of 5.0, 7.9, 10.9 and 13.7. The calibrants are designed to recreate HbA1c variability in red blood cells. The calibrant set also enables determination of the lowest limit of quantification of the assay.

A calibrant bead may serve as a positive or a negative control. A negative control bead may comprise a non-target biomolecule, such as bovine serum albumin, and therefore may provide a baseline signal, such as an estimate of non-specific fluorescent antibody binding to a bead. A positive control bead (e.g., comprising 96% pure HbA1c protein) may serve as a calibrant, and further may enable quantitation of a target analyte. A composition, method, or kit may provide a plurality of calibrant beads comprising differing levels of a target analyte. A composition, method, or kit may provide a positive control bead and/or a negative control bead.

### Example 4: Detection of Labels and Biomolecules by Fluorosequencing

Biomolecules, such as a peptide, that are immobilized on capture beads are identified through fluorosequencing. Briefly, the process involves the following: (1) Labeling of amino acids on peptides with fluorophores: Based on the chemical reactivity of the side chain of amino acids, distinct fluorophores are installed. Each amino acid type is labeled with a select fluorophore, and the fluorophores have mutually distinguishable fluorescence properties. The C-terminal carboxylic acid is modified selectively to form an alkyne moiety. (2) The fluorescently labeled peptides are then released from the beads and immobilized on an azide functionalized glass slide using through copper assisted click chemistry. (3) Following the immobilization of the peptides on the glass slide, fluorescent peptides are imaged at single molecule resolution using Total Internal Reflection Fluorescence (TIRF) microscopy optics. (4) Edman degradation chemistry is performed through fluidic lines, involving cycles of base incubation (20% phenylisothiocyanate (v/v in pyridine) for 40 minutes at 40° C., followed by incubation in TFA for 30 minutes at 40° C. Each cycle cleaves the N-terminal amino acid from the peptide backbone. (5) Following every cycle of Edman degradation, the objective scans the glass slide acquiring images for 1000s of fields and generating intensity profiles for millions of individual peptide spots. (6) Image processing and computational analysis is performed to identify the positions of the fluorescently amino acids, thereby identifying a fluorosequence for every individual peptide. Matching the fluorosequence to a database of reference peptides confirms the identity of the peptide and allows for quantification of peptides in the mixture.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and/or illustrations of the embodiments herein are not meant to be construed in a limiting sense. Numerous variations, changes, and/or substitutions will now occur to those skilled in the art without departing from the invention. Furthermore, it shall be understood that all aspects of the invention are not limited to the specific depictions, configurations or relative proportions set forth herein which depend upon a variety of conditions and/or variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EMBODIMENTS

The following non-limiting embodiments provide illustrative examples of the invention, but do not limit the scope of the invention.

Embodiment 1. A method comprising:
a. contacting a cell and a support in a droplet, wherein the cell comprises at least one biomolecule, wherein the contacting forms a conjugate comprising the support and the at least one biomolecule;
b. collecting the conjugate into a container;
c. labeling the at least one biomolecule of the conjugate in the container with at least one detectable moiety to produce at least one labeled biomolecule; and
d. detecting the at least one labeled biomolecule.

Embodiment 2. The method of embodiment 1, wherein the droplet comprises one cell and one support.

Embodiment 3. The method of embodiment 1 or 2, wherein the droplet comprises an aqueous solution.

Embodiment 4. The method of any one of embodiments 1-3, wherein the droplet is a water-in-oil droplet.

Embodiment 5. The method of any one of embodiments 1-4, wherein the droplet has a mean volume of from about 0.5 pL to about 750 pL.

Embodiment 6. The method of any one of embodiments 1-5, wherein the droplet has a mean volume of from about 100 pL to about 200 pL.

Embodiment 7. The method of any one of embodiments 1-6, wherein the droplet further comprises a cell lysis solution.

Embodiment 8. The method of any one of embodiments 1-7, wherein the droplet further comprises a hypotonic lysis solution.

Embodiment 9. The method of embodiment 7, wherein the cell lysis solution comprises a polyoxyethylene sorbitol ester.

Embodiment 10. The method of embodiment 7, wherein the cell lysis solution comprises 2-[4-(2,4,4-trimethylpentan-2-yl)phenoxy]ethanol.

Embodiment 11. The method of embodiment 7, wherein the cell lysis solution comprises sodium dodecyl sulphate.

Embodiment 12. The method of any one of embodiments 1-11, wherein the droplet further comprises a buffer.

Embodiment 13. The method of embodiment 12, wherein the buffer comprises sodium phosphate.

Embodiment 14. The method of embodiment 12, wherein the buffer comprises tris(hydroxymethyl)aminomethane.

Embodiment 15. The method of embodiment 12, wherein the buffer comprises phosphate-buffered saline.

Embodiment 16. The method of any one of embodiments 1-15, wherein the at least one biomolecule is a polypeptide.

Embodiment 17. The method of any one of embodiments 1-15, wherein the at least one biomolecule is a protein.

Embodiment 18. The method of any one of embodiments 1-15, wherein the at least one biomolecule is an intracellular peptide.

Embodiment 19. The method of any one of embodiments 1-15, wherein the at least one biomolecule is an intracellular protein.

Embodiment 20. The method of any one of embodiments 1-15, wherein the at least one biomolecule is a secreted peptide.

Embodiment 21. The method of any one of embodiments 1-15, wherein the at least one biomolecule is a secreted protein.

Embodiment 22. The method of any one of embodiments 1-15, wherein the at least one biomolecule is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), an oxidized protein, and a glycosylated protein.

Embodiment 23. The method of any one of embodiments 1-15, wherein the at least one biomolecule is hemoglobin Ale (HbA1c).

Embodiment 24. The method of any one of embodiments 1-23, wherein the cell is a red blood cell.

Embodiment 25. The method of any one of embodiments 1-24, wherein the support is a bead.

Embodiment 26. The method of embodiment 25, wherein the bead is a magnetic bead.

Embodiment 27. The method of embodiment 25, wherein the bead comprises a mean diameter of from about 15 nm to about 5 μm.

Embodiment 28. The method of embodiment 25, wherein the bead comprises a mean diameter of from about 1 μm to about 5 μm.

Embodiment 29. The method of embodiment 25, wherein the bead comprises a capture moiety.

Embodiment 30. The method of embodiment 29, wherein the capture moiety comprises pyridine carboxyaldehyde.

Embodiment 31. The method of embodiment 29, wherein the capture moiety comprises a maleimide.

Embodiment 32. The method of embodiment 29, wherein the capture moiety comprises a haloacetyl group.

Embodiment 33. The method of embodiment 29, wherein the capture moiety comprises a pyridyl disulfide.

Embodiment 34. The method of embodiment 25, wherein the bead comprises a plurality of capture moieties.

Embodiment 35. The method of embodiment 34, wherein the plurality of capture moieties comprises pyridine carboxyaldehyde.

Embodiment 36. The method of embodiment 34, wherein the plurality of capture moieties comprises a maleimide group.

Embodiment 37. The method of embodiment 34, wherein the plurality of capture moieties comprises a haloacetyl group.

Embodiment 38. The method of embodiment 34, wherein the plurality of capture moieties comprises a pyridyl disulfide.

Embodiment 39. The method of embodiment 25, wherein the bead comprises a barcode.

Embodiment 40. The method of embodiment 39, wherein the barcode is a deoxyribonucleic acid (DNA), a ribonucleic acid (RNA), a peptide nucleic acid (PNA), a polypeptide, or a combination thereof.

Embodiment 41. The method of embodiment 39, wherein the barcode is an oligopeptide comprising from about 2 amino acids to about 30 amino acids.

Embodiment 42. The method of embodiment 39, wherein the barcode comprises glutamic acid, aspartic acid, cysteine, methionine, lysine, arginine, tyrosine, tryptophan, and phenylalanine.

Embodiment 43. The method of embodiment 39, wherein the barcode comprises a non-natural amino acid.

Embodiment 44. The method of embodiment 39, wherein the barcode is identified with nanopore sequencing.

Embodiment 45. The method of embodiment 39, wherein the barcode is identified with tandem mass spectrometry.

Embodiment 46. The method of embodiment 41, further comprising sequencing the oligopeptide barcode molecule with a method that comprises Edman degradation.

Embodiment 47. The method of any one of embodiments 1-46, wherein the conjugate comprises a disulfide bond.

Embodiment 48. The method of any one of embodiments 1-46, wherein the conjugate comprises a thioether bond.

Embodiment 49. The method of any one of embodiments 1-48, wherein the collecting comprises centrifugation.

Embodiment 50. The method of any one of embodiments 1-49, wherein the collecting comprises applying a magnetic field to the support, wherein the support is a magnetic bead.

Embodiment 51. The method of any one of embodiments 1-49, wherein the collecting comprises adding a reagent, wherein the reagent releases the conjugate from the droplet.

Embodiment 52. The method of embodiment 51, wherein the reagent is an organic solvent.

Embodiment 53. The method of embodiment 52, wherein the organic solvent is isopropanol.

Embodiment 54. The method of any one of embodiments 1-53, wherein the container is a well.

Embodiment 55. The method of any one of embodiments 1-53, wherein the container is a tube.

Embodiment 56. The method of any one of embodiments 1-55, wherein the labeling comprises immunostaining.

Embodiment 57. The method of any one of embodiments 1-56, wherein the detectable moiety comprises a fluorescent moiety.

Embodiment 58. The method of embodiment 57, wherein the fluorescent moiety comprises an antibody.

Embodiment 59. The method of embodiment 57, wherein the fluorescent moiety comprises a fluorescent dye.

Embodiment 60. The method of embodiment 57, wherein the fluorescent moiety comprises Alexa Fluor 488.

Embodiment 61. The method of embodiment 57, wherein the fluorescent moiety comprises Atto647N.

Embodiment 62. The method of any one of embodiments 1-61, wherein the labeling produces a plurality of labeled biomolecules.

Embodiment 63. The method of any one of embodiments 1-62, wherein the detecting comprises flow cytometry.

Embodiment 64. The method of any one of embodiments 1-62, wherein the detecting comprises sequencing by degradation.

Embodiment 65. A method comprising:
(a) contacting a cell and a support in a droplet,
   wherein the cell comprises at least one biomolecule, wherein the at least one biomolecule comprises a first reactive moiety;
   wherein the support comprises a second reactive moiety;
   wherein the first reactive moiety and the second reactive moiety form a covalent bond to form a conjugate comprising the support and the at least one biomolecule;
(b) collecting the conjugate into a container;
(c) labeling the at least one biomolecule with at least one detectable moiety to produce at least one labeled biomolecule; and
(d) detecting the at least one labeled biomolecule.

Embodiment 66. The method of embodiment 65, wherein the droplet comprises one cell and one support.

Embodiment 67. The method of embodiment 65 or 66, wherein the droplet comprises an aqueous solution.

Embodiment 68. The method of any one of embodiments 65-67, wherein the droplet is a water-in-oil droplet.

Embodiment 69. The method of any one of embodiments 65-68, wherein the droplet has a mean volume of from about 0.5 pL to about 750 pL.

Embodiment 70. The method of any one of embodiments 65-69, wherein the droplet has a mean volume of from about 100 pL to about 200 pL.

Embodiment 71. The method of any one of embodiments 65-70, wherein the droplet further comprises a cell lysis solution.

Embodiment 72. The method of any one of embodiments 65-71, wherein the droplet further comprises a hypotonic lysis solution.

Embodiment 73. The method of embodiment 71, wherein the cell lysis solution comprises a polyoxyethylene sorbitol ester.

Embodiment 74. The method of embodiment 71, wherein the cell lysis solution comprises 2-[4-(2,4,4-trimethylpentan-2-yl)phenoxy]ethanol.

Embodiment 75. The method of embodiment 71, wherein the cell lysis solution comprises sodium dodecyl sulphate.

Embodiment 76. The method of any one of embodiments 65-75, wherein the droplet further comprises a buffer.

Embodiment 77. The method of embodiment 76, wherein the buffer comprises sodium phosphate.

Embodiment 78. The method of embodiment 76, wherein the buffer comprises tris(hydroxymethyl)aminomethane.

Embodiment 79. The method of embodiment 76, wherein the buffer comprises phosphate-buffered saline.

Embodiment 80. The method of any one of embodiments 65-79, wherein the at least one biomolecule is a polypeptide.

Embodiment 81. The method of any one of embodiments 65-79, wherein the at least one biomolecule is a protein.

Embodiment 82. The method of any one of embodiments 65-79, wherein the at least one biomolecule is an intracellular peptide.

Embodiment 83. The method of any one of embodiments 65-79, wherein the at least one biomolecule is an intracellular protein.

Embodiment 84. The method of any one of embodiments 65-79, wherein the at least one biomolecule is a secreted peptide.

Embodiment 85. The method of any one of embodiments 65-79, wherein the at least one biomolecule is a secreted protein.

Embodiment 86. The method of any one of embodiments 65-79, wherein the at least one biomolecule is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), an oxidized protein, and a glycosylated protein.

Embodiment 87. The method of any one of embodiments 65-79, wherein the at least one biomolecule is hemoglobin A1c (HbA1c).

Embodiment 88. The method of any one of embodiments 65-87, wherein the cell is a red blood cell.

Embodiment 89. The method of any one of embodiments 65-88, wherein the support is a bead.

Embodiment 90. The method of embodiment 89, wherein the bead is a magnetic bead.

Embodiment 91. The method of embodiment 89, wherein the bead comprises a barcode.

Embodiment 92. The method of embodiment 91, wherein the barcode is a deoxyribonucleic acid (DNA), a ribonucleic acid (RNA), a peptide nucleic acid (PNA), a polypeptide, or a combination thereof.

Embodiment 93. The method of embodiment 91, wherein the barcode is an oligopeptide comprising from about 2 amino acids to about 30 amino acids.

Embodiment 94. The method of embodiment 91, wherein the barcode comprises glutamic acid, aspartic acid, cysteine, methionine, lysine, arginine, tyrosine, tryptophan, and phenylalanine.

Embodiment 95. The method of embodiment 91, wherein the barcode comprises a non-natural amino acid.

Embodiment 96. The method of embodiment 91, wherein the barcode is identified with nanopore sequencing.

Embodiment 97. The method of embodiment 91, wherein the barcode is identified with tandem mass spectrometry.

Embodiment 98. The method of embodiment 93, further comprising sequencing the oligopeptide barcode molecule with a method that comprises Edman degradation.

Embodiment 99. The method of embodiment 89, wherein the bead comprises a mean diameter of from about 15 nm to about 5 μm.

Embodiment 100. The method of embodiment 89, wherein the bead comprises a mean diameter of from about 1 μm to about 5 μm.

Embodiment 101. The method of any one of embodiments 65-100, wherein the first reactive moiety comprises a sulfhydryl group.

Embodiment 102. The method of any one of embodiments 65-100, wherein the first reactive moiety comprises a cysteine side chain.

Embodiment 103. The method of any one of embodiments 65-100, wherein the first reactive moiety comprises a histidyl side chain.

Embodiment 104. The method of any one of embodiments 65-103, wherein the second reactive moiety comprises a pyridine carboxyaldehyde.

Embodiment 105. The method of any one of embodiments 65-103, wherein the second reactive moiety comprises a maleimide group.

Embodiment 106. The method of any one of embodiments 65-103, wherein the second reactive moiety comprises a haloacetyl group.

Embodiment 107. The method of any one of embodiments 65-103, wherein the second reactive moiety comprises a pyridyl disulfide.

Embodiment 108. The method of any one of embodiments 65-107, wherein the support comprises a plurality of second reactive moieties.

Embodiment 109. The method of any one of embodiments 65-108, wherein the covalent bond is a disulfide bond.

Embodiment 110. The method of any one of embodiments 65-108, wherein the covalent bond is a thioether bond.

Embodiment 111. The method of any one of embodiments 65-110, wherein the collecting comprises applying a magnetic field to the support, wherein the support is a magnetic bead.

Embodiment 112. The method of any one of embodiments 65-111, wherein the collecting comprises adding a reagent, wherein the reagent releases the conjugate from the droplet.

Embodiment 113. The method of embodiment 112, wherein the reagent is an organic solvent.

Embodiment 114. The method of embodiment 113, wherein the organic solvent is isopropanol.

Embodiment 115. The method of any one of embodiments 65-114, wherein the container is a well.

Embodiment 116. The method of any one of embodiments 65-114, wherein the container is a tube.

Embodiment 117. The method of any one of embodiments 65-116, wherein the labeling comprises immunostaining.

Embodiment 118. The method of any one of embodiments 65-117, wherein the detectable moiety comprises a fluorescent moiety.

Embodiment 119. The method of embodiment 118, wherein the fluorescent moiety comprises an antibody.

Embodiment 120. The method of embodiment 118, wherein the fluorescent moiety comprises a fluorescent dye.

Embodiment 121. The method of embodiment 118, wherein the fluorescent moiety comprises Alexa Fluor 488.

Embodiment 122. The method of embodiment 118, wherein the fluorescent moiety comprises Atto647N.

Embodiment 123. The method of any one of embodiments 65-122, wherein the labeling produces a plurality of labeled biomolecules.

Embodiment 124. The method of any one of embodiments 65-123, wherein the detecting comprises flow cytometry.

Embodiment 125. The method of any one of embodiments 65-123, wherein the detecting comprises fluorosequencing.

Embodiment 126. The method of any one of embodiments 65-123, wherein the detecting comprises sequencing by degradation.

Embodiment 127. A method comprising:
(a) contacting a cell and a support in a droplet, wherein the cell comprises at least one polypeptide, wherein the contacting forms a conjugate comprising the support and the at least one polypeptide;
(b) labeling the at least one polypeptide with at least one detectable moiety to produce at least one labeled polypeptide; and
(c) detecting the at least one labeled polypeptide using sequencing by degradation.

Embodiment 128. The method of embodiment 127, wherein the droplet comprises one cell and one support.

Embodiment 129. The method of embodiment 127 or 128, wherein the droplet comprises an aqueous solution.

Embodiment 130. The method of any one of embodiments 127-129, wherein the droplet is a water-in-oil droplet.

Embodiment 131. The method of any one of embodiments 127-130, wherein the droplet has a mean volume of from about 0.5 pL to about 750 pL.

Embodiment 132. The method of any one of embodiments 127-131, wherein the droplet has a mean volume of from about 100 pL to about 200 pL.

Embodiment 133. The method of any one of embodiments 127-132, wherein the droplet further comprises a cell lysis solution.

Embodiment 134. The method of any one of embodiments 127-133, wherein the droplet further comprises a hypotonic lysis solution.

Embodiment 135. The method of embodiment 133, wherein the cell lysis solution comprises a polyoxyethylene sorbitol ester.

Embodiment 136. The method of embodiment 133, wherein the cell lysis solution comprises 2-[4-(2,4,4-trimethylpentan-2-yl)phenoxy]ethanol.

Embodiment 137. The method of embodiment 133, wherein the cell lysis solution comprises sodium dodecyl sulphate.

Embodiment 138. The method of any one of embodiments 127-137, wherein the droplet further comprises a buffer.

Embodiment 139. The method of embodiment 138, wherein the buffer comprises sodium phosphate.

Embodiment 140. The method of embodiment 138, wherein the buffer comprises tris(hydroxymethyl)aminomethane.

Embodiment 141. The method of embodiment 138, wherein the buffer comprises phosphate-buffered saline.

Embodiment 142. The method of any one of embodiments 127-141, wherein the at least one polypeptide is an intracellular peptide.

Embodiment 143. The method of any one of embodiments 127-141, wherein the at least one polypeptide is an intracellular protein.

Embodiment 144. The method of any one of embodiments 127-141, wherein the at least one polypeptide is a secreted peptide.

Embodiment 145. The method of any one of embodiments 127-141, wherein the at least one polypeptide is a secreted protein.

Embodiment 146. The method of any one of embodiments 127-141, wherein the at least one polypeptide is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), an oxidized protein, and a glycosylated protein.

Embodiment 147. The method of any one of embodiments 127-141, wherein the at least one polypeptide is hemoglobin A1c (HbA1c).

Embodiment 148. The method of any one of embodiments 127-147, wherein the cell is a red blood cell.

Embodiment 149. The method of any one of embodiments 127-148, wherein the support is a bead.

Embodiment 150. The method of embodiment 149, wherein the bead is a magnetic bead.

Embodiment 151. The method of embodiment 149, wherein the bead comprises a barcode.

Embodiment 152. The method of embodiment 151, wherein the barcode is a deoxyribonucleic acid (DNA), a ribonucleic acid (RNA), a peptide nucleic acid (PNA), a polypeptide, or a combination thereof.

Embodiment 153. The method of embodiment 151, wherein the barcode is an oligopeptide comprising from about 2 amino acids to about 30 amino acids.

Embodiment 154. The method of embodiment 151, wherein the barcode comprises glutamic acid, aspartic acid, cysteine, methionine, lysine, arginine, tyrosine, tryptophan, and phenylalanine.

Embodiment 155. The method of embodiment 151, wherein the barcode comprises a non-natural amino acid.

Embodiment 156. The method of embodiment 151, wherein the barcode is identified with nanopore sequencing.

Embodiment 157. The method of embodiment 151, wherein the barcode is identified with tandem mass spectrometry.

Embodiment 158. The method of embodiment 153, further comprising sequencing the oligopeptide barcode molecule with a method that comprises Edman degradation.

Embodiment 159. The method of embodiment 149, wherein the bead comprises a mean diameter of from about 15 nm to about 5 μm.

Embodiment 160. The method of embodiment 149, wherein the bead comprises a mean diameter of from about 1 μm to about 5 μm.

Embodiment 161. The method of embodiment 149, wherein the bead comprises a capture moiety.

Embodiment 162. The method of embodiment 161, wherein the capture moiety comprises pyridine carboxyaldehyde.

Embodiment 163. The method of embodiment 161, wherein the capture moiety comprises a maleimide group.

Embodiment 164. The method of embodiment 161, wherein the capture moiety comprises a haloacetyl group.

Embodiment 165. The method of embodiment 161, wherein the capture moiety comprises a pyridyl disulfide.

Embodiment 166. The method of embodiment 149, wherein the bead comprises a plurality of capture moieties.

Embodiment 167. The method of embodiment 166, wherein the plurality of capture moieties comprises pyridine carboxyaldehyde.

Embodiment 168. The method of embodiment 166, wherein the plurality of capture moieties comprises a maleimide group.

Embodiment 169. The method of embodiment 166, wherein the plurality of capture moieties comprises a haloacetyl group.

Embodiment 170. The method of embodiment 166, wherein the plurality of capture moieties comprises a pyridyl disulfide.

Embodiment 171. The method of any one of embodiments 127-170, wherein the conjugate comprises a disulfide bond.

Embodiment 172. The method of any one of embodiments 127-170, wherein the conjugate comprises a thioether bond.

Embodiment 173. The method of any one of embodiments 127-172, wherein the collecting comprises centrifugation.

Embodiment 174. The method of any one of embodiments 127-172, wherein the collecting comprises applying a magnetic field to the support, wherein the support is a magnetic bead.

Embodiment 175. The method of any one of embodiments 127-174, wherein the collecting comprises adding a reagent, wherein the reagent releases the conjugate from the droplet.

Embodiment 176. The method of embodiment 175, wherein the reagent is an organic solvent.

Embodiment 177. The method of embodiment 176, wherein the organic solvent is isopropanol.

Embodiment 178. The method of any one of embodiments 127-177, wherein the container is a well.

Embodiment 179. The method of any one of embodiments 127-177, wherein the container is a tube.

Embodiment 180. The method of any one of embodiments 127-179, wherein the labeling comprises immunostaining.

Embodiment 181. The method of any one of embodiments 127-180, wherein the detectable moiety comprises a fluorescent moiety.

Embodiment 182. The method of embodiment 181, wherein the fluorescent moiety comprises an antibody.

Embodiment 183. The method of embodiment 181, wherein the fluorescent moiety comprises a fluorescent dye.

Embodiment 184. The method of embodiment 181, wherein the fluorescent moiety comprises Alexa Fluor 488.

Embodiment 185. The method of embodiment 181, wherein the fluorescent moiety comprises Atto647N.

Embodiment 186. The method of any one of embodiments 127-185, wherein the labeling produces a plurality of labeled polypeptides.

Embodiment 187. A method comprising:
(a) sorting a biological sample to extract a subset of cells;
(b) contacting at least one biomolecule from the subset of cells and a support in a droplet, wherein the cell comprises the at least one biomolecule, wherein the contacting forms a conjugate comprising the support and the at least one biomolecule;
(c) after the contacting, collecting the conjugate into a container;
(d) after the collecting, labeling the at least one biomolecule in the container with at least one detectable moiety to produce at least one labeled biomolecule; and
(e) detecting the at least one detectable moiety.

Embodiment 188. The method of embodiment 187, wherein the droplet comprises one cell and one support.

Embodiment 189. The method of embodiment 187 or 188, wherein the droplet comprises an aqueous solution.

Embodiment 190. The method of any one of embodiments 187-189, wherein the droplet is a water-in-oil droplet.

Embodiment 191. The method of any one of embodiments 187-190, wherein the droplet has a mean volume of from about 0.5 pL to about 750 pL.

Embodiment 192. The method of any one of embodiments 187-191, wherein the droplet has a mean volume of from about 100 pL to about 200 pL.

Embodiment 193. The method of any one of embodiments 187-192, wherein the droplet further comprises a cell lysis solution.

Embodiment 194. The method of any one of embodiments 187-193, wherein the droplet further comprises a hypotonic lysis solution.

Embodiment 195. The method of embodiment 193, wherein the cell lysis solution comprises a polyoxyethylene sorbitol ester.

Embodiment 196. The method of embodiment 193, wherein the cell lysis solution comprises 2-[4-(2,4,4-trimethylpentan-2-yl)phenoxy]ethanol.

Embodiment 197. The method of embodiment 193, wherein the cell lysis solution comprises sodium dodecyl sulphate.

Embodiment 198. The method of any one of embodiments 187-197, wherein the droplet further comprises a buffer.

Embodiment 199. The method of embodiment 198, wherein the buffer comprises sodium phosphate.

Embodiment 200. The method of embodiment 198, wherein the buffer comprises tris(hydroxymethyl)aminomethane.

Embodiment 201. The method of embodiment 198, wherein the buffer comprises phosphate-buffered saline.

Embodiment 202. The method of any one of embodiments 187-201, wherein the at least one biomolecule is a polypeptide.

Embodiment 203. The method of any one of embodiments 187-201, wherein the at least one biomolecule is a protein.

Embodiment 204. The method of any one of embodiments 187-201, wherein the at least one biomolecule is an intracellular peptide.

Embodiment 205. The method of any one of embodiments 187-201, wherein the at least one biomolecule is an intracellular protein.

Embodiment 206. The method of any one of embodiments 187-201, wherein the at least one biomolecule is a secreted peptide.

Embodiment 207. The method of any one of embodiments 187-201, wherein the at least one biomolecule is a secreted protein.

Embodiment 208. The method of any one of embodiments 187-201, wherein the at least one biomolecule is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), an oxidized protein, and a glycosylated protein.

Embodiment 209. The method of any one of embodiments 187-201, wherein the at least one biomolecule is hemoglobin Alc (HbA1c).

Embodiment 210. The method of any one of embodiments 187-209, wherein the cell is a red blood cell.

Embodiment 211. The method of any one of embodiments 187-210, wherein the support is a bead.

Embodiment 212. The method of embodiment 211, wherein the bead is a magnetic bead.

Embodiment 213. The method of embodiment 211, wherein the bead comprises a barcode.

Embodiment 214. The method of embodiment 213, wherein the barcode is a deoxyribonucleic acid (DNA), a ribonucleic acid (RNA), a peptide nucleic acid (PNA), a polypeptide, or a combination thereof.

Embodiment 215. The method of embodiment 213, wherein the barcode is an oligopeptide comprising from about 2 amino acids to about 30 amino acids.

Embodiment 216. The method of embodiment 213, wherein the barcode comprises glutamic acid, aspartic acid, cysteine, methionine, lysine, arginine, tyrosine, tryptophan, and phenylalanine.

Embodiment 217. The method of embodiment 213, wherein the barcode comprises a non-natural amino acid.

Embodiment 218. The method of embodiment 213, wherein the barcode is identified with nanopore sequencing.

Embodiment 219. The method of embodiment 213, wherein the barcode is identified with tandem mass spectrometry.

Embodiment 220. The method of embodiment 215, further comprising sequencing the oligopeptide barcode molecule with a method that comprises Edman degradation.

Embodiment 221. The method of embodiment 211, wherein the bead comprises a mean diameter of from about 15 nm to about 5 μm.

Embodiment 222. The method of embodiment 211, wherein the bead comprises a mean diameter of from about 1 μm to about 5 μm.

Embodiment 223. The method of embodiment 211, wherein the bead comprises a capture moiety.

Embodiment 224. The method of embodiment 223, wherein the capture moiety comprises pyridine carboxyaldehyde.

Embodiment 225. The method of embodiment 223, wherein the capture moiety comprises a maleimide group.

Embodiment 226. The method of embodiment 223, wherein the capture moiety comprises a haloacetyl group.

Embodiment 227. The method of embodiment 223, wherein the capture moiety comprises a pyridyl disulfide.

Embodiment 228. The method of embodiment 211, wherein the bead comprises a plurality of capture moieties.

Embodiment 229. The method of embodiment 228, wherein the plurality of capture moieties comprises pyridine carboxyaldehyde.

Embodiment 230. The method of embodiment 228, wherein the plurality of capture moieties comprises a maleimide group.

Embodiment 231. The method of embodiment 228, wherein the plurality of capture moieties comprises a haloacetyl group.

Embodiment 232. The method of embodiment 228, wherein the plurality of capture moieties comprises a pyridyl disulfide.

Embodiment 233. The method of any one of embodiments 187-232, wherein the conjugate comprises a disulfide bond.

Embodiment 234. The method of any one of embodiments 187-232, wherein the conjugate comprises a thioether bond.

Embodiment 235. The method of any one of embodiments 187-234, wherein the collecting comprises centrifugation.

Embodiment 236. The method of any one of embodiments 187-234, wherein the collecting comprises applying a magnetic field to the support, wherein the support is a magnetic bead.

Embodiment 237. The method of any one of embodiments 187-234, wherein the collecting comprises adding a reagent, wherein the reagent releases the conjugate from the droplet.

Embodiment 238. The method of embodiment 237, wherein the reagent is an organic solvent.

Embodiment 239. The method of embodiment 238, wherein the organic solvent is isopropanol.

Embodiment 240. The method of any one of embodiments 187-239, wherein the container is a well.

Embodiment 241. The method of any one of embodiments 187-239, wherein the container is a tube.

Embodiment 242. The method of any one of embodiments 187-241, wherein the labeling comprises immunostaining.

Embodiment 243. The method of any one of embodiments 187-242, wherein the detectable moiety comprises a fluorescent moiety.

Embodiment 244. The method of embodiment 243, wherein the fluorescent moiety comprises an antibody.

Embodiment 245. The method of embodiment 243, wherein the fluorescent moiety comprises a fluorescent dye.

Embodiment 246. The method of embodiment 243, wherein the fluorescent moiety comprises Alexa Fluor 488.

Embodiment 247. The method of embodiment 243, wherein the fluorescent moiety comprises Atto647N.

Embodiment 248. The method of any one of embodiments 187-247, wherein the labeling produces a plurality of labeled biomolecules.

Embodiment 249. The method of any one of embodiments 187-248, wherein the detecting comprises flow cytometry.

Embodiment 250. The method of any one of embodiments 187-248, wherein the detecting comprises sequencing by degradation.

Embodiment 251. A composition comprising: (a) at least one polypeptide; and (b) a singular support, wherein the at least one biomolecule and the singular support are coupled together by a covalent bond, and wherein the at least one biomolecule and the singular support are enclosed in a droplet.

Embodiment 252. The composition of embodiment 251, wherein the droplet comprises one cell and one support.

Embodiment 253. The composition of embodiment 251 or 252, wherein the droplet comprises an aqueous solution.

Embodiment 254. The composition of any one of embodiments 251-253, wherein the droplet is a water-in-oil droplet.

Embodiment 255. The composition of any one of embodiments 251-254, wherein the droplet has a mean volume of from about 0.5 pL to about 750 pL.

Embodiment 256. The composition of any one of embodiments 251-255, wherein the droplet has a mean volume of from about 100 pL to about 200 pL.

Embodiment 257. The composition of any one of embodiments 251-256, wherein the droplet further comprises a cell lysis solution.

Embodiment 258. The composition of any one of embodiments 251-257, wherein the droplet further comprises a hypotonic lysis solution.

Embodiment 259. The composition of embodiment 257, wherein the cell lysis solution comprises a polyoxyethylene sorbitol ester.

Embodiment 260. The composition of embodiment 257, wherein the cell lysis solution comprises 2-[4-(2,4,4-trimethylpentan-2-yl)phenoxy]ethanol.

Embodiment 261. The composition of embodiment 257, wherein the cell lysis solution comprises sodium dodecyl sulphate.

Embodiment 262. The composition of any one of embodiments 251-261, wherein the droplet further comprises a buffer.

Embodiment 263. The composition of embodiment 262, wherein the buffer comprises sodium phosphate.

Embodiment 264. The composition of embodiment 262, wherein the buffer comprises tris(hydroxymethyl)aminomethane.

Embodiment 265. The composition of embodiment 262, wherein the buffer comprises phosphate-buffered saline.

Embodiment 266. The composition of any one of embodiments 251-265, wherein the at least one biomolecule is a polypeptide.

Embodiment 267. The composition of any one of embodiments 251-265, wherein the at least one biomolecule is a protein.

Embodiment 268. The composition of any one of embodiments 251-265, wherein the at least one biomolecule is an intracellular peptide.

Embodiment 269. The composition of any one of embodiments 251-265, wherein the at least one biomolecule is an intracellular protein.

Embodiment 270. The composition of any one of embodiments 251-265, wherein the at least one biomolecule is a secreted peptide.

Embodiment 271. The composition of any one of embodiments 251-265, wherein the at least one biomolecule is a secreted protein.

Embodiment 272. The composition of any one of embodiments 251-265, wherein the at least one biomolecule is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), an oxidized protein, and a glycosylated protein.

Embodiment 273. The composition of any one of embodiments 251-265, wherein the at least one biomolecule is hemoglobin A1c (HbA1c).

Embodiment 274. The composition of any one of embodiments 251-271, wherein the support is a bead.

Embodiment 275. The composition of any one of embodiments 272, wherein the bead is a magnetic bead.

Embodiment 276. The composition of embodiment 272, wherein the bead comprises a barcode.

Embodiment 277. The composition of embodiment 274, wherein the barcode is a deoxyribonucleic acid (DNA), a ribonucleic acid (RNA), a peptide nucleic acid (PNA), a polypeptide, or a combination thereof.

Embodiment 278. The composition of embodiment 274, wherein the barcode is an oligopeptide comprising from about 2 amino acids to about 30 amino acids.

Embodiment 279. The composition of embodiment 274, wherein the barcode comprises glutamic acid, aspartic acid, cysteine, methionine, lysine, arginine, tyrosine, tryptophan, and phenylalanine.

Embodiment 280. The composition of embodiment 274, wherein the barcode comprises a non-natural amino acid.

Embodiment 281. The composition of embodiment 272, wherein the bead comprises a mean diameter of from about 15 nm to about 5 μm.

Embodiment 282. The composition of embodiment 272, wherein the bead comprises a mean diameter of from about 1 μm to about 5 μm.

Embodiment 283. The composition of any one of embodiments 251-282, wherein the conjugate comprises a disulfide bond.

Embodiment 284. The composition of any one of embodiments 251-282, wherein the conjugate comprises a thioether bond.

Embodiment 285. The composition of any one of embodiments 251-282, wherein the conjugate further comprises a fluorescent moiety.

Embodiment 286. The composition of embodiment 285, wherein the fluorescent moiety comprises an antibody.

Embodiment 287. The composition of embodiment 285, wherein the fluorescent moiety comprises a fluorescent dye.

Embodiment 288. The composition of embodiment 285, wherein the fluorescent moiety comprises Alexa Fluor 488.

Embodiment 289. The composition of embodiment 285, wherein the fluorescent moiety comprises Atto647N.

Embodiment 290. A method comprising:
(a) providing a droplet comprising a cell and a bead, wherein the cell comprises a polypeptide;
(b) permeabilizing the cell within the droplet, thereby bringing the polypeptide in contact with the bead, wherein upon the polypeptide coming in contact with the bead, the polypeptide is coupled to the bead;
(c) releasing the bead having the polypeptide coupled thereto from the droplet; and
(d) identifying the polypeptide while still coupled to the bead.

Embodiment 291. A method comprising:
(a) providing a droplet comprising a cell and a bead, wherein the cell comprises a polypeptide;
(b) permeabilizing the cell within the droplet, thereby bringing the polypeptide in contact with the bead, wherein upon the polypeptide coming in contact with the bead, the polypeptide is coupled to the bead;
(c) releasing the bead having the polypeptide coupled thereto from the droplet; and
(d) identifying the polypeptide using sequencing by degradation.

Embodiment 292. A method comprising:
(a) contacting a cell and a support in a droplet, wherein the cell comprises at least one biomolecule;
(b) labeling the at least one biomolecule; and
(c) detecting a level of post-translational modification of the cell by detecting the at least one labeled biomolecule by sequencing by degradation.

1. A method comprising:
(a) contacting a cell and a support in a droplet, wherein said cell comprises at least one biomolecule, wherein said at least one biomolecule comprises a first reactive moiety; wherein said support comprises a second reactive moiety; wherein said first reactive moiety and said second reactive moiety form a covalent bond to form a conjugate comprising said support and said at least one biomolecule;
(b) collecting said conjugate into a container;
(c) labeling said at least one biomolecule with at least one detectable moiety to produce at least one labeled biomolecule; and
(d) detecting said at least one labeled biomolecule.

2. The method of claim 1, wherein said droplet comprises one cell and one support.

3. The method of claim 1, wherein said droplet is a water-in-oil droplet.

4. The method of claim 1, wherein said droplet has a mean volume of from about 0.5 pL to about 750 pL.

5-6. (canceled)

7. The method of claim 1, wherein said at least one biomolecule is a polypeptide.

8. The method of claim 1, wherein said at least one biomolecule is an intracellular peptide or a secreted peptide.

9. (canceled)

10. The method of claim 1, wherein said at least one biomolecule is an intracellular protein or a secreted protein.

11. The method of claim 1, wherein said at least one biomolecule is selected from the group consisting of lactic acid, nicotinamide, 5-oxoproline, xanthine, hypoxanthine, glucose, malic acid, adenine, hemoglobin A (HbA), hemoglobin A2 (HbA2), hemoglobin A1 (HbA1), hemoglobin F (HbF), an oxidized protein, and a glycosylated protein.

12. (canceled)

13. The method of claim 1, wherein said support is a bead.

14. The method of claim 13, wherein said bead comprises a barcode.

15. (canceled)

16. The method of claim 14, wherein said barcode comprises a non-natural amino acid.

17-20. (canceled)

21. The method of claim 1, wherein said second reactive moiety comprises a pyridine carboxyaldehyde.

22-24. (canceled)

25. The method of claim 1, wherein said support comprises a plurality of second reactive moieties.

26-27. (canceled)

28. The method of claim 1, wherein said collecting comprises adding a reagent, wherein said reagent releases said conjugate from said droplet.

29. (canceled)

30. The method of claim 1, wherein said detectable moiety comprises a fluorescent moiety.

31. The method of claim 30, wherein said fluorescent moiety comprises an antibody.

32. The method of claim 30, wherein said fluorescent moiety comprises a fluorescent dye.

33. The method of claim 1, wherein said labeling produces a plurality of labeled biomolecules.

34. The method of claim 1, wherein said detecting comprises sequencing by degradation.

35. A method comprising:
(a) contacting a cell and a support in a droplet, wherein said cell comprises at least one polypeptide, wherein said contacting forms a conjugate comprising said support and said at least one polypeptide;
(b) labeling said at least one polypeptide with at least one detectable moiety to produce at least one labeled polypeptide; and
(c) detecting said at least one labeled polypeptide using sequencing by degradation.

36-62. (canceled)

63. A method comprising:
(a) providing a droplet comprising a cell and a bead, wherein said cell comprises a polypeptide;
(b) permeabilizing said cell within said droplet, thereby bringing said polypeptide in contact with said bead, wherein upon said polypeptide coming in contact with said bead, said polypeptide is coupled to said bead;
(c) releasing said bead having said polypeptide coupled thereto from said droplet; and
(d) identifying said polypeptide while still coupled to the bead.

64. A method comprising:
(a) providing a droplet comprising a cell and a bead, wherein said cell comprises a polypeptide;
(b) permeabilizing said cell within said droplet, thereby bringing said polypeptide in contact with said bead, wherein upon said polypeptide coming in contact with said bead, said polypeptide is coupled to said bead;
(c) releasing said bead having said polypeptide coupled thereto from said droplet; and
(d) identifying said polypeptide using sequencing by degradation.

65. A method comprising:
(a) contacting a cell and a support in a droplet, wherein said cell comprises at least one biomolecule;
(b) labeling said at least one biomolecule; and
(c) detecting a level of post-translational modification of said cell by detecting said at least one labeled biomolecule by sequencing by degradation.
